(19) United States
(12) Patent Application Publication
(10) Pub. No.: US 2023/0341392 A1
(43) Pub. Date: Oct. 26, 2023

(54) DETECTION OF ANTIBODIES TO SARS-COV2

(71) Applicant: IDEXX LABORATORIES, INC., Westbrook, ME (US)

(72) Inventors: Jesse Stephen Buch, Kennebunk, ME (US); Joshua Michael Boucher, Gray, ME (US); Jiayou Liu, Scarborough, ME (US); Trevor Justin Morin, Scarborough, ME (US); Eugene Regis Krah, III, Yarmouth, ME (US)

(21) Appl. No.: 18/008,551

(22) PCT Filed: Jun. 8, 2021
(86) PCT No.: PCT/IB21/00395

§ 371 Date: Dec. 6, 2022

Related U.S. Application Data
(60) Provisional application No. 63/036,403, filed on Jun. 8, 2020.

(51) Int. Cl. G01N 33/569; G01N 33/533

(52) U.S. Cl. CPC G01N 33/56983; G01N 33/533; G01N 2469/20; G01N 2333/165

(57) ABSTRACT

The disclosure is directed to methods and apparatus for the detecting, diagnosis of infection with SARS-CoV-2. The disclosure includes methods for detecting a current or former SARS-CoV-2 infection in an animal, methods for diagnosing and treating an animal infected with SARS-CoV-2. in various aspects, the disclosure is directed to the detection of a nucleocapsid polypeptide of SARS-CoV-2 in a sample from an animal, the detection of a portion of a spike polypeptide of SARS-CoV-2 in a sample from and animal, or detection of both a portion of a spike polypeptide of SARS-CoV-2 and a portion of a nucleocapsid polypeptide of SARS-CoV-2 in a sample from an animal.

### RELATED APPLICATIONS

This application claims the benefit of U.S., Provisional Patent Application No. 63/036,403, filed Jun. 8, 2020, which is incorporated by reference herein in its entirety.

### SEQUENCE LISTING STATEMENT

A computer readable form of the Sequence Listing is filed with this application by electronic submission and is incorporated into this application by reference in its entirety. The Sequence Listing is contained in the file created on Jun. 8, 2021, having the file name “20-950-WO_Sequence-Listing_SEQ.txt” and is 58 kb in size.

### FIELD

The disclosure generally relates to the determination of infection of an animal with a SARS-CoV-2 virus.

### BACKGROUND

After a person is infected with SARS-CoV-2, the virus that causes the coronavirus (Covid-19) disease, that person's immune system will produce antibodies against the virus to fight the infection. The SARS-CoV-2 virus has four structural proteins known as the S (spike), E (envelope), M (membrane), and N (nucleocapsid) proteins, After infection with the virus, a person's immune system will produce antibodies that specifically bind to portions of the M, S, N and E proteins to help the cells of the immune system to fight and rid the body of the virus. The antibodies circulate throughout the body so, that they can bind to, and help get rid of, the virus. Currently, it is believed that it takes about 8 to 10 days after being infected with the virus for the body to produce enough antibodies to be detected in a blood sample. It is known that the antibodies remain in the body for an extended period of time, possibly the entire lifetime of the animal, but the exact amount of time is currently under investigation.

In response to the worldwide Covid-19 pandemic, there is a need in the art for identifying animals that have been infected with BARS-CoV-2, and there is a further need for sensitive and specific assays that can identify antibodies in a sample from the animal to indicate whether an animal is currently or has been previous infected with the virus

### SUMMARY

This Summary lists several embodiments of the presently disclosed subject manor, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, disclosure is directed to a method for detecting a current or former SARS-CoV-2 infection in an animal, including determining a presence or amount of an antibody that binds to a portion of a nucleocapsid polypeptide of SARS-CoV-2 in a sample from the animal, determining a presence or amount of an antibody that binds to a portion of a spike polypeptide of SARS-CoV-2 in the sample, and determining that the animal has a current or has had a previous SARS-CoV-2 infection by determining in the sample the presence or amount of at least one of the antibody that binds to a portion of nucleocapsid polypeptide and/or at least one the antibody that binds to the portion of the spike polypeptide.

In some embodiments, the nucleocapsid polypeptide comprises SEQ ID NO:83, and m some embodiments, the portion of the nucleocapsid comprises at least three consecutive amino acids from the RNA binding domain of the nucleocapsid polypeptide (SEQ ID NO:96, or at least three consecutive amino acids from SEQ NOS: 6-14, 17-24, 70-82, 86-88, or 99-106.

In some embodiments, the spike polypeptide comprises SEQ ID NO:84, and in some embodiments the portion of the spike polypeptide comprises at least three amino acids from a receptor binding domain of the spike polypeptide, which in some embodiments comprises SEQ ID NO 85.

Embodiments of the disclosure are also directed to a device for determining a current or former SARS-CoV-2 infection in an animal, including a solid phase having bound thereto a first polypeptide comprising a least a portion of a nucleocapsid polypeptide SARS-CoV-2 and/or a second polypeptide comprising at least a portion of a spike polypeptide of SARS-CoV-2. In some embodiments, the nucleocapsid polypeptide comprises SEQ ID NO:83, and in some embodiments, the portion of the nucleocapsid polypeptide comprises at least three consecutive amino acids from the RNA binding domain of the nucleocapsid polypeptide (SEQ ID NO:96), or at least three consecutive amino acids from one of SEQ ID NOS: 6-14, 17-24, 70-82, 86-88, or 99-106.

In some embodiments, the spike polypeptide comprises SEQ ID NO:84, and in some embodiments, the portion of the spike polypeptide comprises at least three amino acids from a receptor binding domain of the spike polypeptide, which, in embodiment, includes SEQ ID NO:85.

Embodiments of the disclosure are also directed to a kit for determining a current or former SARS-CoV-2 infection in an animal, including the device for determining a current or former SARS-CoV-2 infection in an animal as described herein and above and a conjugate comprising a labeled binding moiety that binds at least one of an antibody that binds to a nucleocapsid polypeptide of SARS-CoV-2 and/or at least one of an antibody that binds to a spike polypeptide of SARS-CoV-2. in some embodiments, the labeled binding moiety includes an anti-species IgG antibody, where the species is that of the animal.

Embodiments of the disclosure are also directed to a kit for determining a current or former SARS-CoV-2 infection in an animal, including a device for determining a current or former SARS-CoV-2 infection in an animal as described herein and above, a first conjugate including a first labeled binding moiety that binds an antibody that binds to a nucleocapsid polypeptide of SARS-CoV-2, and/or a second conjugate including a labeled binding moiety that binds an antibody that binds to a spike polypeptide of SARS-CoV-2. In some embodiments, the first conjugate includes at least a portion of the nucleocapsid polypeptide, which in embodiments includes SEQ ID NO 83. In some embodiments of the kit, the portion of the nucleocapsid polypeptide of the conjugate is the same as the portion of the nucleocapsid polypeptide bound to the solid phase, and in some embodiments, the second conjugate comprises at least a portion of the, spike polypeptide. In some embodiments of the kit, the portion of the spike polypeptide of the conjugate is the same as the portion of the spike polypeptide bound to the solid phase.

Embodiments of the disclosure are also directed to a conjugate including at least consecutive three amino acids from a nucleocapsid polypeptide of SARS-CoV-2 and a detectable label, which nucleocapsidpolypeptide, in some embodiments, includes SEQ ID NO:83. In some embodiments, the at least three amino acids are from the RNA binding domain of the nucleocapsid polypeptide (SEQ ID NO:96), and in some embodiments, the at least three consecutive amino acids are from one of SEQ ID NOS: 6-14, 17-24, 70-82 and 86-88,

In some embodiments, the conjugate includes at least three consecutive three amino acids horn a spike polypeptide of SARS-CoV-2 and a detectable label, and in some embodiments, the three consecutive ammo acids are from the receptor binding domain of the spike polypeptide. In some embodiments, the spike polypeptide includes SEQ ID NO: 84, and in some embodiments, the receptor binding domain comprises SEQ ID NO:85.

Embodiments of the disclosure are also directed to a solid phase having bound to it (a) a first immunological complex including an antibody that binds to a portion of a nucleocapsid polypeptide of SARS-CoV-2 in a sample from the animal and the conjugate of one of claims 22-25, and/or (b) a second immunological complex comprising an antibody that binds to a portion of a spike polypeptide of SARS-CoV-2 in a sample from the animal and the conjugate of one of claims 26-29.

Embodiments of the disclosure are also directed to a polypeptide comprising at least three consecutive amino acids from one of SEQ ID NOS:6-14, 17-24, 70-82, 86-88 and 99-106. In some embodiments, the poly-peptide includes an amino acid sequence selected from the group consisting of any one of SEQ ID NOS:1-82, 86-88 and 99-106, and in some embodiments, the polypeptide includes an amino acid sequence selected from the group consisting of any one of SEQ ID NOS:6-14, 17-24, 70-82, 86-88, and 99-106. In sonic embodiments, the polypeptide includes an amino acid sequence selected from the group consisting of any one of SEQ ID NOS:6-14, and 17-24, and in some embodiments, the polypeptide includes an amino acid sequence selected from the group consisting, of any one of SEQ ID NOS: 70-82, and 86-88. In some embodiments, the polypeptide includes an amino acid sequence selected from the group consisting of any one of SEQ ID NOS:99-106.

In some embodiments, the polypeptide further includes a detectable labels which, in some embodiments, includes one or more of fluorescent label, isotopic label, biotin label, or enzyme conjugate label. In some embodiments, the polypeptide is reversibly or irreversibly bound to a solid support, and in some embodiments, the polypeptide is attached at its N-terminus, its terminus or both termini to one or more other peptide sequences.

Embodiments of the disclosure are also directed to an immune complex including one or more polypeptides as described herein and above and one or more antibodies that specifically binds the one or more polypeptides, which one or more antibodies is from a sample from an animal suspected of having a SARS-CoV-2 infection.

Embodiments of the disclosure are also directed to a method of treating an animal infected with SARS-CoV-2, including determining a presence or amount of an antibody that binds to a portion of a nucleocapsid polypeptide of SARS-CoV-2 in a sample from the animal, determining a presence or amount of an antibody that binds to a portion of a spike polypeptide of SARS-CoV-2 in the sample, determining that the animal has a SARA-CoV-2 infection by determining in the sample the presence or amount: of at least one of the antibody that binds to a portion of nucleocapsid polypeptide or at least one of the antibody that binds to the portion of the spike polypeptide, and administering an effective amount of a pharmaceutical composition to treat the SARS-CoV-2 infection. in some embodiments, the method includes determining in the sample the presence or amount of both an antibody that binds to a portion of nucleocapsid polypeptide and an antibody that binds to the portion of the spike polypeptide.

In some embodiments of the method, the animal has exhibited one or more symptoms of SARS-CoV-2 for no more than about 14, about 13 days, about 12 days, about 11 days, about 10 days, about 9 days, about 8 days, about 7 days, about 6 days, about 5 days, about 4 days, about 3 days, about 2 days, or about 1 day, and in some embodiments, the animal has exhibited one or more symptoms of SARS-CoV-2 for no more than about 10 days.

In some embodiments of the method of treating an animal infected with SARS-CoV-2, the composition includes one of more of antiviral drugs, corticosteroids, convalescent plasma, monoclottal antibodies, interleukin inhibitor's, anti-parasitics, antibiotics, kinase inhibitors, interferons, and anti-inflammatories. In some embodiments, the antiviral drugs include one or more of remdesivir, lopinavir, ritonavir, darunavir, favipiravir, umifenovir, oseltamivir, galidesivir, disulftram, danoprevir or nelfinavir, favipiravir, ribavirin, galidesivir, griffithsin, nafamostat. In some embodiments, anti-parasitics include one or more of hydroxychloroquine, chloroquine, or ivermectin, and in some embodiments, the one or more antibiotics include azithromycin, amoxicillin, clindamycin, cephalexin, ciprofloxacin, sulfamethoxazole/trimethoprim, metronidazole, levofloxacin, and doxycycline.

In some embodiments, monoclonal antibodies include one or more of bamlanivimab, etesevimab, casirivimab, imdevimab, S230.15, m396, S1.00.8 S227.14, S230.15, 80R scFv, CR3022 CR3014, 33G4 35B5, 30F9, 4D4, IF8, 5E9, B1 scFv, 47D11, HA001, B38, H4, or CR3022, and in some embodiments, interleukin inhibitors include one or more of tocilizumab or sarilumab, and in some embodiments, the kinase inhibitors comprise one or more of acalabrutinib, baricitinib, ruxolitinib or tofacitinib.

### BRIEF DESCRIPTION OF THE DRAWINGS

The accompanying drawings, which are included to provide a further understanding of the disclosure, are incorporated in and constitute a part of this specification, illustrate embodiments of the disclosure and together with the detailed description serve to explain the principles of the invention. No attempt is made to show structural details of the invention in more detail than may be necessary for a fundamental understanding of the invention and various ways in which it may be practiced.

FIG. 1 is a schematic diagram of the SARS-CoV-2 spike polypeptide. Superscript denotes E. coli expression constructs TDX1801¹ and TDX1802² respectively.

FIG. 2 is a schematic diagram of the SARS-CoV2 nucleocapsid polypeptide.

FIG. 3 shows the results of an experiment to identify a signal associated with the binding of-anti-SARS-CoV-2 antibodies in a sample to a full-length SARS-CoV-2 spike polypeptide.

FIG. 4 shows the results of an experiment to identify a signal associated with the binding of anti-SARS-CoV-2 antibodies in a sample to a full-length SARS-CoV-2 nucleocapsid polypeptide.

FIG. 5 shows the combination of the signals from certain samples from FIGS. 3 and 4.

FIGS. 6A-F. FIGS. 6A-C (through SEQ ID NO:82) show the results of a screen of 15 amino acid fragments of a SARS-COV-2 nucleocapsid polypeptide for binding to antibodies in a sample from an animal infected with SARS-CoV-2. FIGS. 6C-F show a number of SARS-CoV-2 polypeptide sequences and non-SARS-CoV-2 polypeptides used as controls in examples of some of the methods described herein.

FIG. 7, Panels A and B, show scheematic representations of the immunoassay formats of the disclosure.

FIGS. 8A-E. FIGS. 8A-D show detection of SARS-CoV-2 spike-receptor binding domain (Spike-RBD), nucleocapsid protein (Np) or both Spike-RED and Np antigens by sera from known SARS-CoV2 patients. The dual antigen assay was more sensitive than either single antigen assay. FIG. 8E shows that experimental protein levels for RDB, Np and RDB+Np were essentially equivalent.

FIGS. 9A-B show detection of SARS-CoV-2-specific antibodies in saliva from unvaccinated, partially vaccinated and fully vaccinated donors.

FIG. 10 shows the detection of SARS-CoV-2-specific total antibodies to Spike-RBD in samples from pre-vaccinated, partially vaccinated and fully vaccinated patients.

### DESCRIPTION

The disclosure is directed to immunological methods, devices, reagents, and kits for detecting the presence of an amount of antibodies to SARS-CoV-2 in a biological sample. The methods, kits and devices may include reagents, controls, calibrators or standards including one or more of SARS-COV-2 antigens conjugated to detectable labels. In various aspects, the disclosure is directed to using immunoassay techniques, including, but not limited to, using solid supports (microplates, porous matrices, flow through solid phase matrices, and lateral flow devices) having bound thereto the SARS-CoV-2 antigens that bind to antibodies in the sample. The presence or amount of the antibodies on the solid supports can be detected with the labeled conjugates. Animal subjects from which samples are obtained for detecting antibodies include human and non-human (e.g., companion animals; livestock, etc.) subjects. The determination of disease states, including current or former infection with SARS-CoV-2, which may be associated with the presence or amount of the antibodies, can be conducted for both human and non-human subjects.

Before addressing the various aspects of the disclosure in more detail, a number of terms are defined below.

The term “antigen,” as used herein, generally refers to a substance that is capable, under appropriate conditions, of reacting with an antibody specific for the antigen. For the purposes of this disclosure, antigens include portions of the nucleocapsid and spike polypeptide regions of the SARS-CoV-2 virus as more fully described herein.

The term “analyte,” as used herein, generally refers to the substance, or set of substances in a sample that are detected and/or measured, For the purposes of the present disclosure, anti-SARS-CoV-2 antibodies are analytes.

The term “animal” as used herein, generally refers to any animal, e.g., a human, or a non-human animal companion animals, livestock and animals in the wild.

The term “sample,” as used herein, generally refers to a sample of tissue or fluid from a human or animal including, but not limited to whole blood, plasma, serum, spinal fluid, lymph fluid, abdominal fluid (ascites), the external sections of skin, respiratory, intestinal and genitourinary tracts, tears, saliva, urine, blood cells, tumors, organs, tissue, and sample of in vitro cell culture constituents. Many such samples require processing prior to analysis. Sample includes both raw samples and/or processed samples.

The term “blood sample,” as used herein, generally refers to any blood-derived fluid sample, including but not limited to whole blood, plasma, and serum. To provide serum for use in the methods of the disclosure, one or more serum samples are obtained from the animal subject. The serum samples can be, for example, obtained from the animal subject as blood samples, then separated to provide serum. In certain embodiments, the serum can be measured without separation from blood. As the person of skill in the art will appreciate, a single obtained sample can be divided or otherwise used to do both concentration measurements.

The term “immunoassay,” as used herein, generally refers to a test that employs antibody and antigen complexes to generate a measurable response. An “antibody:antigen complex” may be used interchangeably, with the term “immunological complex.” Immunoassays, in general, include noncompetitive immunoassays, competitive immunoassays, homogeneous immunoassays, and heterogeneous immunoassays. Immunoassays that require separation of bound antibody:antigen complexes are generally referred to as “heterogeneous immunoassays,” and immunoassays that do not require separation of antibody:antigen complexes are generally referred to as “homogeneous immunoassays.”

The term “immunolgical complexes,” as used herein, generally refers to the complexes formed by the binding of antigen and antibody molecules, with or without complement fixation. When one of either the antibody or antigen is labeled, the label is associated with the immune complex as a result of the binding between the antigen and antibody. Therefore, when the antibody is labeled, the label becomes associated with the antigen as a result of the binding. Similarly, when the antigen is labeled (e.g., an analyte analog having a label), the label becomes associated with the antibody as a result of the binding between the antigen and the antibody.

The term “label,” as used herein, refers to a detectable compound or composition, which can be conjugated directly or indirectly (e.g., via covalent or non-covalent means, alone or encapsulated) to a SARS-COV-2 antigen of the disclosure. The label may be detectable by itself (e.g., radioisotope labels, chemiluminescent dye, electrochemical labels, metal chelates, latex particles, or fluorescent labels) or, in the case of an enzymatic label, may catalyze chemical alteration of a substrate compound or composition which is detectable (e.g., enzymes such as horseradish peroxidase, alkaline phosphatase, and the like). The label employed in the current disclosure could be, but is not limited to: alkaline phosphatase; glucose-6-phosphate dehydrogenase (“G6PDH”); horse radish peroxidase (HRP); chemiluminescers such as isoluminol, fluorescers such as fluorescein and rhodamine compounds; ribozymes; and dyes. The label may also be a specific binding molecule which itself may be detectable (e.g., biotin, avidin, streptavidin, digoxigenin, maltose, oligohistidine, 2, 4-dinitrobenzene, phenylarsenate, ssDNA, dsDNA, and the like). The utilization of a label produces a signal that may be detected by means such as detection of electromagnetic radiation or direct visualization, and that can optionally be measured.

The terms “solid support”, “solid phase” and “solid matrix” as used herein, refer to a non-aqueous matrix to which the binding partner of the present disclosure can adhere. Examples of solid supports, solid phases, and solid matrices include supports formed partially or entirely of glass (e.g., controlled pore glass), synthetic and natural polymers, polysaccharides (e.g., agarose), polyacrylamides, polystyrene, polyvinyl alcohols and silicones, chromatographic strips, microtiter polystyrene plates, or any other substances that will allow bound binding partners to be washed or separated from unbound materials. In some embodiments, the solid supports, phases and matrices can be porous. In certain embodiments, depending on the application, the solid support, solid phase and solid matrix can be the well of an assay plate. The solid support solid phase and solid matrix may include an analytical test slide as described in U.S. Patent Publication No. 2014/0315216 which is incorporated herein by reference in its entirety.

The term “particle” or “particles” in connection with the disclosure include, for example, particles of latex, polystyrene, or of other support materials such as silica, agarose, ceramics, glass, polyacrylamides, polymethyl methacrylates, carboxylate modified latex, melamine, and Sepharose. The particles will as in size from about 0.1 microns to about 100 microns, for example about 0.1,, 0.5, 1.0, 5, 10, 20, 30, 40 50, 60, 70, 80, 90 or 100 microns. hi particular, useful commercially available materials include carboxylate modified latex, cyanogen bromide activated Sepharose beads, fused silica particles, isothiocyanate glass, polystyrene, and carboxylate monodisperse microspheres. The particles may be magnetic or paramagnetic. Particles suitable for use in the present invention are capable of attachment to other substances such as derivatives, linker molecules or proteins. The capability of the particles to be attached to other substances can result from the particle material as well as from any surface modifications or functional groups added to the particle. The particles can be functionalized or he capable of becoming functionalized in order to covalently or non-covalently attach proteins, linker molecules or derivatives as described herein. Suitable functional groups include, for example, amine, biotin, streptavidin, avidin, protein A, sulfhydryl, hydroxyl and carboxyl.

“Receptor” refers to any compound or composition capable of recognizing a particular spatial and polar organization of a molecule, e.g., epitopic or determinant site. Illustrative receptors include antibodies, Fab fragments, and the like.

“Binding specificity” or “specific binding” refers to the substantial recognition of a first molecule for a second molecule, for example a polypeptide and a polyclonal or monoclonal antibody, or an antibody fragment (e.g. a Fv single chain Fv, Fab′, or F(ab′)2 fragment) specific for the polypeptide. For example, “specificity,” as used herein, generally refers to the ability of an individual antibody combining site to react with only one antigenic determinant or the ability of a population of antibody molecules to react with only one antigen. In general, there is a high degree of specificity in antigen-antibody reactions. Antibodies can distinguish differences in (i) the primary structure of an antigen, (ii) isomeric forms of an antigen, and (iii) secondary and tertiary structure of an antigen. Antibody-antigen reactions that exhibit high specificity exhibit low cross reactivity.

“Substantial binding” or “substantially bind” refers to an amount of specific binding or recognizing between molecules in an assay mixture under particular assay conditions. In its broadest aspect, substantial binding relates to the difference between a first molecule's incapability of binding or recognizing a second molecule, and the first molecules capability of binding or recognizing a third molecule, such that the difference is sufficient to allow a meaningful assay to be conducted distinguishing specific binding under a particular set of assay conditions, which includes the relative concentrations of the molecules, and the time and temperature of an incubation. In another aspect, one molecule is substantially incapable of binding or recognizing another molecule in a cross-reactivity sense where the first molecule exhibits a reactivity for a second molecule that is less than 25%, less than 10%, less than 5% or less than 1% of the reactivity exhibited toward a third molecule under a particular set of assay conditions. Specific binding can be tested using a number of widely known methods, e.g., an immunohistochemical assay, an enzyme-linked immunosorbent assay (ELISA), a radioimmunoassay (RIA), or a western blot assay.

“Effective amount” refers to an amount sufficient to achieve or at least partially achieve the desired effect. The term “effective dose” is defined as an amount sufficient to cure or at least partially arrest a SARS-CoV infection in a patient already suffering from such infection. Effective amounts for this use will depend upon the severity of the infection and the general state of the patient's own immune system.

Turning now to the various aspects of the disclosure, the disclosure includes methods, devices, reagents and kits for detecting a current or former SARS-CoV-2 infection in an animal. In one aspect, a method includes the following:
determining a presence or amount of an antibody that binds to a portion of a nucleocapsid polypeptide of SARS-CoV-2 in a sample from the animal;
determining a presence or amount of an antibody that binds to a portion of a spike polypeptide of SARS-CoV-2 in the sample; and
determining that the animal has a current or has had a previous SARS-CoV-2 infection by determining in the sample the presence or amount of at least one of the antibody that binds to a portion of nucleocapsid polypeptide or at least one of the antibody that binds to the portion of the spike polypeptide.

In another aspect, a method includes the following:
determining a presence or amount: of an antibody that binds to a portion of a nucleocapsid polypeptide of SARS-CoV-2 in a sample from the animal;
determining a presence or amount of an antibody that binds to a portion of a spike polypeptide of SARS-CoV-2 in the sample; and
determining that the animal has a current or has had a previous SARS-CoV-2 infection by determining in the sample the presence or amount of at least one of the antibody that binds to a portion of nucleocapsid polypeptide and at least one of the antibody that binds to the portion of the spike polypeptide.

That is, determining that the animal has a current or has bad a previous SARS-CoV-2 infection includes determining the presence or amount of at least one antibody that binds to a portion of nucleocapsid-polypeptide and the presence or amount of at least one antibody that binds to a portion of spike polypeptide.

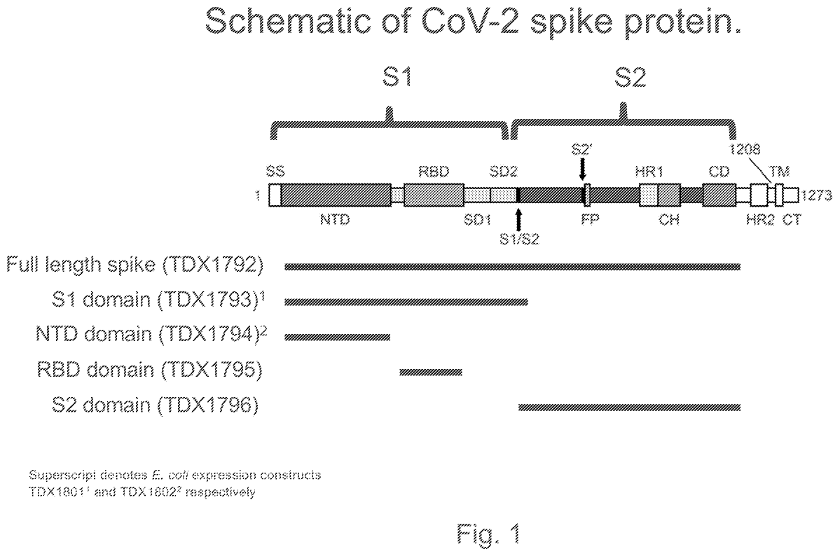

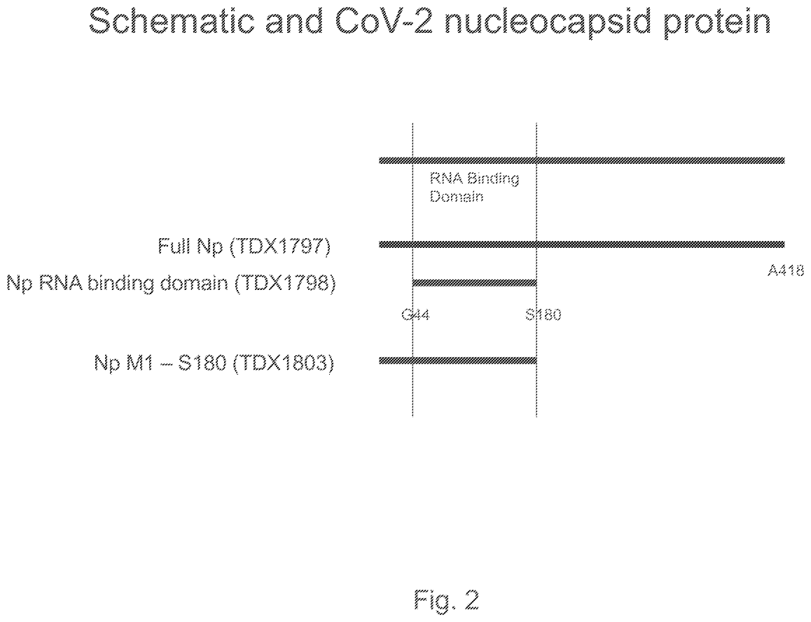

Schematic diagrams of the SARS-CoV-2 nucleocapsid and spike polypeptides are provided in FIGS. 1 and 2. In the methods, devices, reagents and kits of the disclosure, the nucleocapsid polypeptide includes a portion of, includes, or is identical to SEQ ID NO. 83. For instance, the portion of the nucleocapsid polypeptide comprises at least three consecutive amino acids from the RNA binding domain of the nucleocapsid polypeptide (SEQ ID NO:96). For example, the portion of the nucleocapsid polypeptide may include at least 3, at least 4, at least 5, at least 6, at least 7, at least 8, at least 9, at least 10, at least 12, at least 15, or at least 20 consecutive amino acids from SEQ ID NO:83 or SEQ ID NO:96. In addition, the portion of the nucleocapsid polypeptide may be at least three consecutive amino acids from one of SEQ ID NOS:1-82, 86-88 or 99-106, or from SEQ ID NOS:6-14, 17-24, 70-82, 86-88 or 99-106, or comprise the amino acid sequence selected from the group consisting of SEQ ID NOS:1-82, 86-88 and 99-106, or from SEQ ID NOS:6-14 17-24 70-82 86-88 or 99-106, or from SEQ ID NOS:6-14, and 17-24, or from SEQ ID NOS: 70-82, and 86-88, or from SEQ ID NOS:99-106. In some embodiments, the poly-peptide is attached at its N-terminus, its C-terminus or both termini to one or more other peptide sequences.

With regard to the spike polypeptide used, in the various aspects of the disclosure the spike polypeptide includes a portion of, includes, or is identical to SEQ ID NO:84. For instance, a portion of the spike polypeptide may include at least three amino acids from a receptor binding domain of the spike polypeptide (SEQ NO:85), for example at least 3, at least 4, at least 5, at least 6, at least 7, at least 8, at least 9, at least 10, at least 12. at least 15, or at least 20 consecutive amino acids from SEQ ID NO:84 or SEQ ID NO:85.

The disclosure includes a device for determining a current or former SARS-CoV-2 infection in an animal. For example, the device includes a solid phase having bound thereto a first polypeptide comprising a least a portion of a nucleocapsid polypeptide of SARS-CoV-2 or second polypeptide comprising at least a portion of a spike polypeptide of SARS-CoV-2. The device further includes a solid phase having bound thereto a first polypeptide comprising a least a portion of a nucleocapsid polypeptide of SARS-CoV-2 and second polypeptide comprising at least a portion of a spike poly-peptide of SARS-CoV-2. The portions of the nucleocapsid and spike polypeptides are as described above.

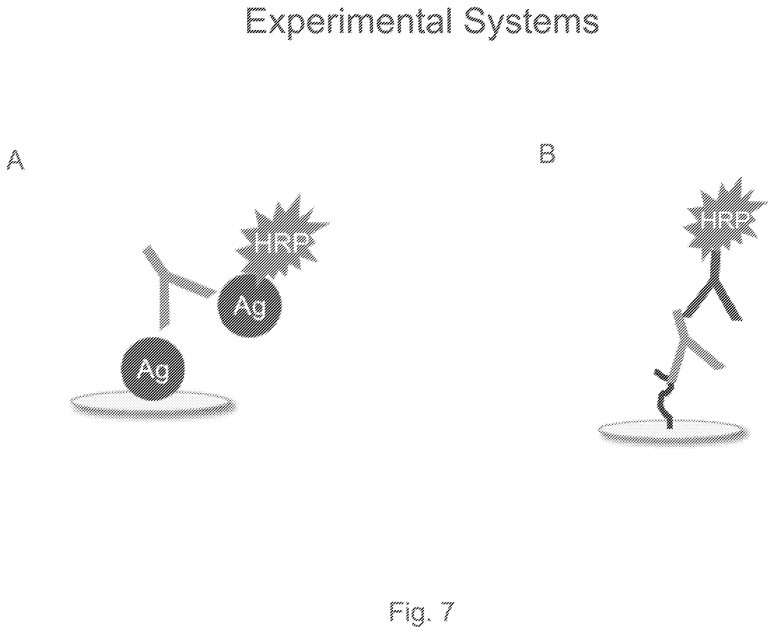

In another aspect, the disclosure includes kits for determining a current or former SARS-CoV-2 infection in an animal. The kits include the devices of the disclosure for capturing antibodies in the sample onto a solid phase and further include reagents to provide a signal related to the presence or amount of the binding of an antibody or antibodies to the solid phase. In one embodiment, the reagents include conjugates of a detectable label attached to a binding moiety that binds the antibodies from the sample that become bound to the solid phase (i.e., an antibody from the sample that binds to a nucleocapsid polypeptide of SARS-CoV-2 and/or an antibody that binds to a spike polypeptide of SARS-CoV-2, which nucleocapsid and spike polypeptides are described herein and e.g., in Table 6). The binding moiety may be an anti-species antibody, for instance an IgG antibody, where the species is that of the animal from which the sample was taken. When the conjugate including the anti-species antibody and the label binds to the antibody or antibodies from the sample captured on a solid phase, the label may be detected to provide a signal corresponding to the presence or amount of an antibody or antibodies in the sample. (See FIG. 7, panel B). When captured antibodies from the swipe are different but the label on the binding moieties is the same, the signal will be present when either of the sample antibodies is captured on the solid phase. The presence of the signal will be indicative of a current or former infection of the animal by SARS-CoV-2.

In another embodiment of a kit according to the disclosure, the kit includes
(a) the device having a solid phase with having bound thereto a first polypeptide comprising a least a portion of a nucleocapsid polypeptide of SARS-CoV-2 and/or second polypeptide comprising at least a portion of a spike polypeptide of SARS-CoV-2,
(b) first conjugate comprising a first labeled binding moiety that binds an antibody that binds to a nucleocapsid polypeptide of SARS-CoV-2, and/or
(c) a second conjugate comprising a labeled binding moiety that binds an antibody that binds to a spike polypeptide of SARS-CoV-2.

In various aspects of the disclosure that are schematically represented in FIG. 7, panel B, the binding moiety of the first conjugate includes a portion of the nucleocapsid polypeptide, which may be the same as the portion of the nucleocapsid polypeptide bound to the solid phase, or at least includes an overlapping portion of the nucleocapsid polypeptide bound to the solid phase such that both, antigens include sufficient portions of the polypeptide sequence (epitope) bound by the antibody from the sample such that the antibody is capable of substantially binding both nucleocapsid antigen sequences. Similarly, the binding moiety of a second conjugate (not shown in panel A) includes at least a portion of the spike polypeptide, which may be the same as the portion of the spike polypeptide bound to the solid phase, or at least includes an overlapping portion of the spike polypeptide bound to the solid phase such that both antigens include sufficient portions of the polypeptide sequence (epitope) bound by the antibody such that the antibody is capable of substantially binding both spike antigen sequences.

Further embodiments of the disclosure include the conjugates described above.

In yet another aspect, the disclosure is directed to solid phase having bound thereto (a) a first immunological complex including an antibody from a biological sample that binds to a portion of a nucleocapsid polypeptide of SARS-CoV-2 from the animal and a conjugate including a nucleocapsid polypeptide as described herein, and (b) a second immunological complex including an antibody that binds to a portion of a spike polypeptide of SARS-CoV-2 its a sample from the animal and a conjugate including a spike polypeptide as described herein.

In various embodiments of the disclosure, the solid phase may include more than one nucleocapsid polypeptides and/or more than one spike polypeptides. Detection reagents including the labeled conjugates for providing a signal when antibodies in the sample are present on the solid phase can be a mixture of labeled conjugates that will bind the antibodies that are captured on the solid phase. The labels on the conjugates may all be the same or may all be different, and the amount of the signal(s) may be determined individually or collectively to determine the presence or amount of antibodies in the sample and a previous or current infection with SARS-CoV-2.

The solid phase assay format is a commonly used binding assay technique. There are a number of assay devices and procedures wherein the presence of an analyte is indicated by the analyte's binding to a conjugate and/or an immobilized complementary binding member. In one particular aspect, the immobilized binding member (e.g., SARS-CoV-2 polypeptide) is bound, or becomes bound during the assay, to a solid phase such as a reaction well, dipstick, test strip, flow-through pad, paper, fiber matrix or other suitable solid phase material. The binding reaction between antibodies in, the sample and immobilized antigen is determined by adding to the sample an amount of conjugate, which includes a binding partner for the antibody conjugated to a label. After contacting the mixture of the sample and the conjugate to the solid phase, the mixture and solid phase are incubated to allow for binding between the antibody in the sample, the antigen on the solid phase, and the conjugate. Following the incubation, unbound reactants are removed from the solid phase. The amount of the label that becomes associated with the solid phase is measured.

Immobilization of one or more SARS-CoV-2 antigens onto a device or solid support is performed so that the antigens will not be washed away by the sample, diluent and/or wash procedures. One or more antigens can be attached to a surface by physical adsorption without the use of chemical linkers) or by chemical binding (i.e., with the use of chemical linkers). Chemical binding can generate stronger attachment of antibodies on a surface and provide defined orientation and confirmation of the surface-bound molecules. Numerous methods of non-diffusively binding polypeptides to solid supports are known. E.g., Immunochemical Protocols; Methods in Molecular Biology; Vol. 295, edited by R. Burns (2005).

Detection of the label associated with the antibody:antigen complexes bound to the solid phase may be achieved through a variety of techniques well known in the art, depending on the label, such as, for example, enzymatic labeling, radiolabeling, luminescence, or fluorescence. Immunoassay methodologies are known by those of ordinary skill in the art and are appreciated to include, but not limited to, radioimmunoassay (RIA), enzyme, immunoassays (EIA), fluorescence polarization immunoassays (FPIA), microparticle enzyme immunoassays (MEIA), enzyme multiplied it technology (EMIT) assays, immunoturbidometric or agglutination assays, colloidal gold-based immunoassays including lateral flow devices and chemiluminescent magnetic immunoassays (CMIA). In EIA, an antibody or antigen is labeled with an enzyme that converts a substrate to a product with a resulting signal that is measured, such as a change in color. In MEIA, a solid phase microparticle is used to capture the analyte. In CMIA, a chemiluminescent label is conjugated to the antigen, and produces light when combined with its substrate. The concentration of analyte measured maybe proportional to the amount of signal measured.

The use of reagent-impregnated test strips, in specific binding assays is also well-known. In such procedures, a test sample is applied to one portion of the test strip and is allowed to migrate or wick through the strip material. Thus, the analyte to be detected or measured passes through or along the material, possibly with the aid of an eluting solvent which can be the test sample itself or a separately added solution. The analyte migrates into a capture or detection zone on the test strip, wherein a complementary binding member to the analyte is immobilized. The extent to which the analyte becomes bound in the detection zone can be determined with the aid of the conjugate which can also be incorporated in the test strip or which can be applied separately. In one embodiment, an antigen specific for SARS-CoV-2 antibodies is immobilized on a solid support at a distinct, location. Following addition of the sample, detection of SARS-CoV-2-antibody complexes on the solid support can be by any means known in the art. For example, U.S. Pat. No. 5,726,010 which is incorporated herein by reference in its entirety, describes an example of a lateral flow device, the SNAP® immunoassay device (IDEXX Laboratories).

Other detection technologies employ magnetic particles or microbeads, for example, superparamagnetic iron oxide impregnated polymer beads. These beads are associated with, for example, a specific binding partner for the analyte. The beads bind with the target analytes in the sample being tested and are then typically isolated or separated out of solution magnetically. Once isolation has occurred, other testing may be conducted, including observing particular images or labels (e.g., a barcode), whether directly optically or by means of a camera.

The SARS-COV-2 antigens described herein may be linked to a label to provide a detectable conjugate for use in receptor binding assays, such as immunoassays that detect SARS-COV-2 antibodies. The SARS-COV-2 antigens can be linked to a label or a solid phase using methods well known to those skilled in the art. E.g., Immunochemical Protocols; Methods in Molecular Biology, Vol. 295 edited b R. Burns (2005).

For each of the spike and nucleocapsid polypeptide sequences described herein, the sequence may be comprised in a longer polypeptide, natural or synthetic, or the polypeptides may consist only of the identified amino acids. The polypeptides may be attached to the solid phases or labels using various forms of chemical and/or polypeptide linkers such that the sequences are available for antibody recognition. Polypeptide linkers may be, for instance, non-immunogenic and may be of any length to the extent the linker does not interfere with antibody binding. For example, linkers of 1-12 amino acids, in particular, 1, 2, 3, 4, 5, 6, 7, 8, 9, 11, or 12 amino acids, may be used. Chemical linkers of 2-40 atoms (counting, for instance, from the terminal amino or carboxyl group of the spike or nucleocapsid polyeptide through the shortest number of atoms to a function group of the solid support or label), are typical (e.g., 2, 3, 4, 5, 6, 7, 8, 9, 10, 11, 12, 15, 20, 25, 30, 35 or 40 atoms).

In other embodiments the immunoassay methodologies are competitive immunoassays for detection of anti-SARS-COV-2 antibodies. The competitive immunoassay may be carried out in the following illustrative manner. A sample, from an animal's body fluid, potentially containing anti-SARS-COV-2 antibodies, is contacted with a SARS-COV-2 analog conjugated to a solid support and with an anti-SARS-COV-2 antibody conjugated to a detectable label. The anti-SARS-COV-2 antibodies of interest, present in the sample, compete with the anti-SARS-COV-2 antibody conjugated to a detectable label for binding with the BARS-COV-2 analog conjugated to a solid support. The amount of the label associated with the solid support can be determined after separating unbound antibodies and the solid support. In an alternative embodiment, the competitive immunoassay is carried out in the following illustrative manner. A sample, from an animal's body fluid, potentially containing anti-SARS-COV-2 antibodies, is contacted with a SARS-COV-2 antigen linked to a detectable label and then with an antibody conjugated to a solid support. The anti-SARS-COV-2 antibodies in the sample compete with the anti-SARS-COV-2 antibodies on the solid support for binding with the SARS-COV-2 conjugate linked to a detectable label. In either case, the signal obtained is inversely related to the amount of SARS-COV-2 antibody of interest present in the sample.

In yet another aspect, the disclosure is directed to a method of treating an animal infected with SARS-CoV-2, including:
determining a presence or amount of an antibody that binds to a portion of a nucleocapsid polypeptide of SARS-CoV-2 in a sample from the animal,
determining a presence or amount of an antibody that binds to a portion of a spike polypeptide of SARS-CoV-2 in the sample;
determining that the animal has a SARS-CoV-2 infection by determining in the sample the presence or amount of at least one of the antibody that binds to a portion of nucleocapsid polypeptide and/or at least one the antibody that binds to the portion of the spike polypeptide; and
administering an effective amount of a pharmaceutical composition to treat the SARS-CoV-2 infection.

In some embodiments, only the presence or amount of an antibody that binds to a portion of a nucleocapsid polypeptide of SARS-CoV-2 in a sample from the animal is determined before determining that the animal has a SARS-CoV-2 infection. In other embodiments, only the presence or amount of an antibody that binds to a portion of a spike polypeptide of SARS-CoV-2 in a sample from the animal is determined before determining that the animal has a SARS-CoV-2 infection, while in other embodiments the presence or amount of an antibody to a portion of a spike polypeptide and antibody to a portion of a nucleocapsid polypeptide are both determined prior to determining that the animal has a SARS-CoV-2 infection.

In some embodiments, the animal is newly infected with SARS-CoV-2, having exhibited one or more symptoms of SARS-CoV-2 for no more than about 14 days, e.g., about 10 days. In some embodiments, the animal has exhibited one or more symptoms for about 13 days, about 12 days, about 11 days, about 10 days, about 9 days, about 8 days, about 7 days, about 6 days, about 5 days, about 4 days, about 3 days, about 2 days, or about 1 day.

Methods of treating an animal infected with SARS-CoV-2 comprise administering to an animal a therapeutically effective amount of a pharmaceutical composition to treat the SARS-CoV-2 infection. In some embodiments, the composition comprises one or more of antivirals, corticosteroids, convalescent plasma, monoclonal antibodies, interleukin inhibitors, anti-parasitics, antibiotics, kinase inhibitors, interferons, anti-inflammatories and combinations thereof, e.g., hydroxychloroquine and azithromycin.

In embodiments, the antiviral drugs comprise one or more of remdesivir, lopinavir, ritonavir, darunavir, favipiravir, umifenovir, oseltamivir, galidesivir, disulfiram, danoprevir or nelfinavir, favipiravir, ribavirin, galidesivir, griffithsin, and nafamostat.

In embodiments, the anti-parasitic comprise one or more of hydroxychloroquine, chloroquine, and ivermectin.

In embodiments, the antibiotic comprise one or more of azithromycin, amoxicillin, clindamycin, cephalexin, ciprofloxacin, sulthmethoxazole/trimethoprim, metronidalzole, levofloxacin, and doxycycline. In some embodiments, the antibiotic comprises azithromycin.

In embodiments, the monoclonal antibodies can comprise one or more of bamlanivimab, etesevimab, casirivimab, imdevimab, S230.15, m396, S109.8 S227.14, S230.15, 80R scFv, CR3022 CR3014, 33G4 35B5, 30F9, 4D4, IF8, 5E9, B1 scFv, 47D11, HA001, B38, H4, and CR3022. Monoclonal antibodies are also described under other specific medicinal categories, e.g., tocilizumab and sarilumab under interleukin inhibitors.

In embodiments, the interleukin inhibitors comprise one or more of interleukin-1 inhibitors and interleukin-6 inhibitors. In embodiments, the interleukin-6 inhibitors include tocilizumab and sarilumab, and the interleukin-1inhibitors include anakinra, canakinumab, and rilonacept.

In embodiments, the kinase inhibitors comprise one or more of acalabrutinib, baricitinib, tofacitinib, acalabrutinib, ibrutinib, and zanubrutinib.

In some embodiments of the various aspects of the disclosure, the one or more pharmaceutical compositions are administered in one dose or in two or more doses. One of skill in the art can determine pharmacokinetic and pharmacodynamic characteristics of a particular pharmaceutical composition that determine whether more than one dose is preferable to a single dose.

In some embodiments, the pharmaceutical composition(s) are administered on multiple occasions. Intervals between single dosages can be intraday, on successive or non-successive days, weekly or monthly. Intervals can also be irregular as indicated by measuring blood level of the virus or of the titer of antibodies generated against the virus.

The pharmaceutical compositions can be administered by oral, paremeral, topical, intravenous, subcutaneous, intraarterial, intracranial, intraperitoneal, intranasal, intraocular or intramuscular means for prophylactic and/or therapeutic treatment. Intramuscular injection is most typically performed in the arm or muscles. Intramuscular injection or intravenous infusion are preferred for administration of antibodies.

In embodiments of the various aspects of the disclosure, the effective amount of pharmaceutical composition can be about 0.01 mg to about 1000 mg, about 0.01 mg to about 900 mg, about 0.01 mg to about 800 mg, about 0.01 mg to about 700 mg, about 0.01 mg to about 600 mg, about 0.01 mg to about 500 mg, about 0.01 mg to about 400 mg, about 0.01 mg to about 300 mg, about 0.01 mg to about 200 mg, about 0.01 mg to about 100 mg, 0.1 mg to about 1000 mg, about 0.1 mg to about 900 mg, about 0.1 mg to about 800 mg, about 0.1 mg to about 700 mg, about 0.1 mg to about 600 mg, about 0.1 mg to about 500 mg, about 0.1 mg to about 400 mg, about 0.1 mg to about 300 mg, about 0.1 mg to about 200 mg, about 0.1 mg to about 100 mg, about 1 mg to about 1000 mg, about 1 mg to about 900 mg, about 1 mg to about 800 mg, about 1 mg to about 700 mg, about 1 mg to about 600 mg, about 1 mg to about 500 mg, about 1 mg to about 400 mg, about 1 mg to about 300 mg, about 1 mg to about 200 mg, about 1 mg to about 100 mg, about 10 mg to about 1000 mg, about 50 mg to about 1000 mg, about 100 mg to about 1000 mg, about 200 mg to about 1000 mg, about 300 mg to about 1000 mg, about 400 mg to about 1000 mg, about 500 mg to about 1000 mg, about 10 mg to about 500 mg, about 50 mg to about 500 mg, about 100 mg to about 500 mg, about 10 mg to about 300 ma, about 50 mg to about 300 mg, from about 100 mg to about 300 mg, about 10 mg to about 150 mg, about 50 mg to about 150 mg, about 60 mg to about 120 mg, about 50 mg to about 120 mg or a range between any two of these values. Specific examples include, for example, about 1000 mg, about 900 mg, about 800 mg, about 700 mg, about 750 mg, about 600 mg, about 500 mg, about 400 mg, about 450 mg, about 300 mg, about 250 mg, about 200 mg, about 175 mg, about 150 mg, about 125 mg, about 120 mg, about 110 mg, about 100 mg, about 90 mg, about 80 mg, about 70 mg, about 60 mg, about 50 mg, about 30 mg, about 20 mg, about 10 mg, about 5 mg, about 1 mg, about 0.1 mg, about 0.01 mg, or any between the ranges disclosed above.

In embodiments, an effective amount of antibody can be from about 0.5 to 300 mg/kg of antibody per dose, with dosages of from about 5 to 25 mg/kg being more commonly used.

In some embodiments, an effective amount can vary according to, for example, the particular use for which the treatment is made, the manner of administration of the compound or composition, the health and condition of the human or non-human annual, and the judgment of the prescribing physician. The proportion or concentration of a compound or composition in a pharmaceutical composition comprising, e.g., one or more of antivirals, corticosteroids, monoclonal antibodies, interleukin inhibitors, anti-parasitics, antibiotics, kinase inhibitors, interferons, anti-inflammatories and combinations thereof can vary depending upon a number of factors including chemical characteristics (e.g., hydrophobicity), and the route of administration. For example, the compounds or compositions can be provided in an aqueous physiological buffer solution containing about 0.1 to about 10% w/v of the compound or composition for parenteral administration. Some typical dose ranges for the compounds or compositions are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the infection, the overall health status of the particular animal, the relative biological efficacy of the compound or composition selected, formulation of the excipient, and its route of administration. Effective doses can be extrapolated from dose-response curves derived from in vitro or animal model test systems.

In embodiments, convalescent plasma is administered intravenously, and one or more units of plasma (typically 200-250 mL) is administered.

In some embodiments, the methods may include the co-administration (concurrent, coincident or sequential administration) of two or more of the antivirals, corticosteroids, convalescent plasma, monoclonal antibodies, interleukin inhibitors, anti-parasitics, antibiotics, kinase inhibitors, interferons, and anti-inflammatories. In embodiments, co-administration of the second pharmaceutical composition may be at the same time, substantially the same time, before or after administration of the first pharmaceutical composition.

In embodiments, the method of treating an animal infected with SARS-CoV-2 acts as an adjuvant prior to, with, or after one or more additional therapies including oxygen therapy and respiratory support (e.g., non-invasive ventilation, high-flow nasal cannula, intubation with active ventilation).

### Uses

Each of the peptides, polypeptides, immunogens, and pharmaceutical compositions described herein may be for use in treating SARS-CoV-2 and/or related viral infections as described herein. In addition, each of the peptides, polypeptides, immunogens, and pharmaceutical compositions described herein ma be for use in methods for treating SARS-CoV-2 and/or related infections as described herein. Each of the peptides, polypeptides, immunogens, and pharmaceutical compositions described herein may be used in a method for manufacturing a medicament for treating or use in treating SARS-CoV-2 and/or related viral infections as described herein.

The following are provided for exemplification purposes only and are not intended to limit the scope of the invention described in broad terms above. All references cited in this disclosure are incorporated herein by reference.

### EXAMPLES

### Example 1

### Simultaneous Detection of SARS-CoV-2-Specific Antibodies to both Spike-RBD and Nucleocapsid Targets

Forty-six (n=46) SARS-CoV-2-positive serum samples were obtained from patients that tested either PCR-positive prior to collection or antibody positive on the day of collection. Ten (n=10) SARS-CoV-2-negative serum samples were obtained from samples collected prior to 2019 when the SARS-CoV-2 virus was not known to be circulating among human populations.

Microtiter plates were pre-coated with either full length recombinant receptor binding domain of the Spike protein (Spike-RBD) or full length recombinant Nucleocapsid protein (Np). ELISA detection reagents comprised matching antigen (either Spike-RBD or Np) conjugated to horseradish peroxidase (HRP). The assay protocol included the following steps: (1) mixing sample with conjugate and incubating on coated plates for one hour to create the double-antigen:antibody sandwich complex diagramed in FIG. 7, panel A; (2) washing and aspirating plates to remove unbound reagents: (3) adding TMD substrate and incubate for 15 minutes; (4) adding acid stop and reading the plate at 450 nm to measure color development in each well.

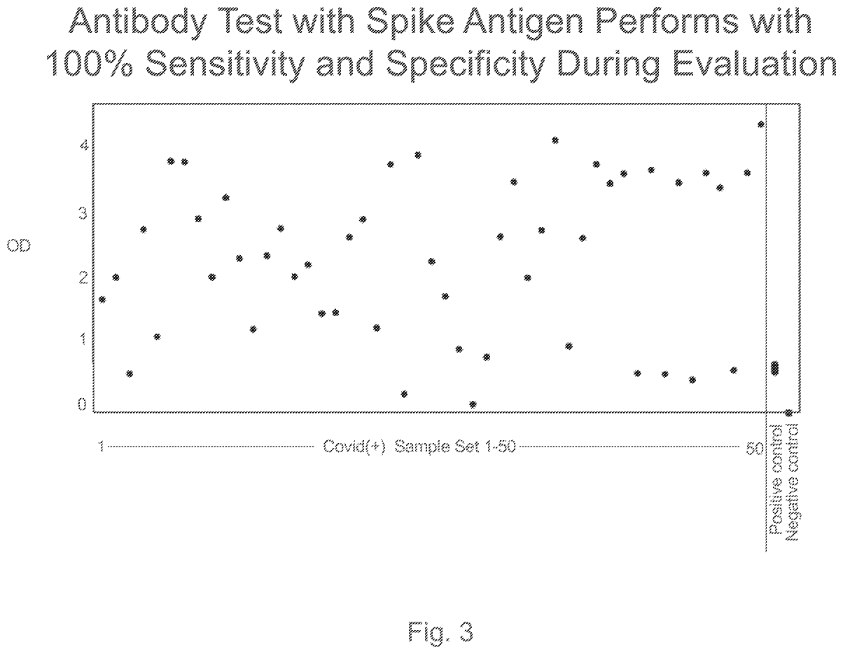

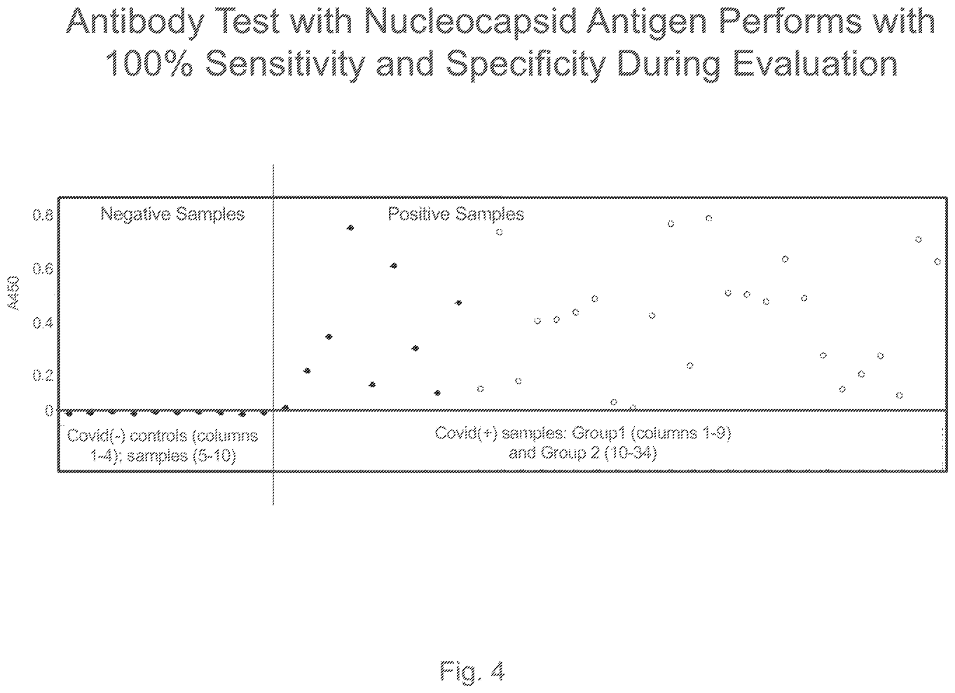

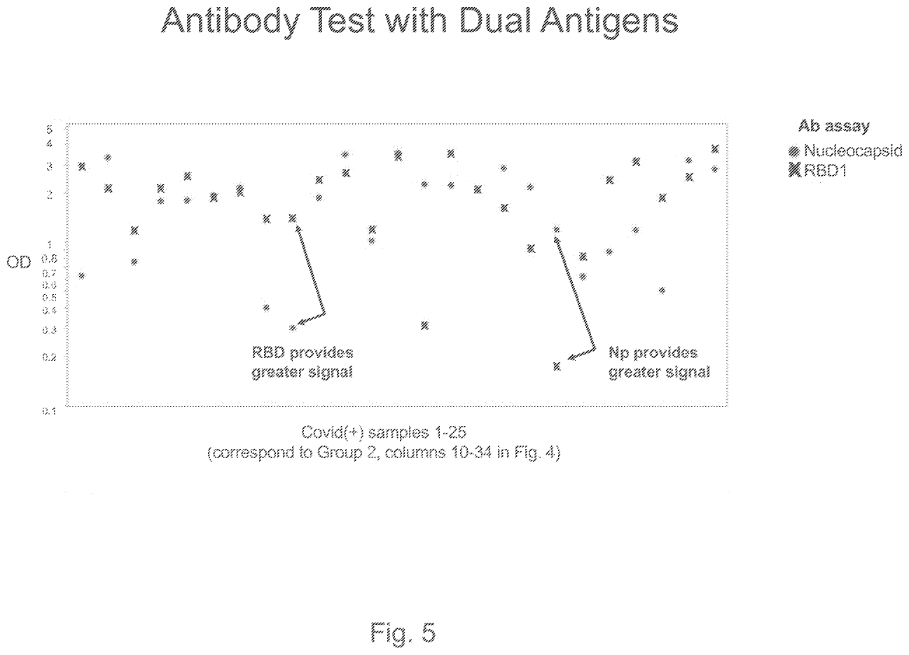

The results (FIGS. 3, 4 and 5) demonstrate that individuals infected with the SARS-CoV-2 virus, the causative agent of the respiratory disease COVID-19, may produce variable antibody responses to different antigenic proteins of the virus. In this experiment, some infected individuals produced a stronger antibody response to Nucleocapsid protein (Np) antigen while other individuals produced a stronger antibody response to the receptor binding domain of the Spike protein (Spike-RBD). These results show that detecting SARS-Cov-2-specific antibodies simultaneously to more than one target (i.e., Spike-RBD+Np) is more sensitive than detecting antibodies to a single target (i.e. either Spike-RBD or Np alone).

### Example 2

### Identification of Linear Immunodominant Epitopes on SARS-CoV-2 Nucleocapsid by Peptide Array

To identify linear immunodominant epitopes coat SARS-CoV-2 Nucleocapsid that bind naturally occurring anti-SARS-CoV-2 antibodies in COVID-19 patient sera were determined using a peptide array.

Sera of five patients each from three categories were pooled: COVID-19 PCR negative (Neg Pool): sera collected <14 days following positive COVID-19 PCR test (Early PCR Pos Pool); sera collected ≥14 days following positive COVID-19 PCR test (Late PCR Pos Pool).

Peptide Array Design: Eighty-two peptides were synthesized to cover the entire length of the SARS-CoV-2 Nucleocapsid protein (M1-A419; Genbank Accession: QHD43423) (SEQ ID NO:83). All peptides in the array were 15 amino acids in length with a 10 amino acid overlap between adjacent peptides. The peptide array included six additional negative control 15-mer sequences, three from Human serum albumin (SEQ ID NOS:93-95), and one each from common Human coronaviruses 229E, OC43, and NL63 (SEQ ID NOS: 90-92)

Assay: Microtiter plate wells were pre-coated separately with the individual array peptides and full length recombinant biotinylated Nucleocapsid protein (Np) as a positive control (SEQ ID NO:89). Goat, anti-human IgG (Fc-specific) HRP was used as the detection reagent, FIG. 7, panel B shows a representation of the immunocomplex formed in the presence of peptide-specific anti-SARS-CoV-2 antibodies. The assay protocol included the following steps: (1) Diluting each sample pool 1:100 and incubating on coated plates for 45 mins; (2) washing and aspirating plates to remove unbound reagents; (3) adding anti-species HRP conjugate and incubating for 45 mins; (4) washing and aspirating plates to remove unbound reagents; (5) adding TMB (3,3′,5,5′-tetramethylbenzidine) substrate and incubating for 15 mins; (6) add acid stop and read plate at 450 nm to measure color development in each well,

SEQ ID NOS:85 and 89 were expressed, isolated and purified utilizing a C-terminal HIS-tag with a TEV protease cleavage site according to methods known in the art.

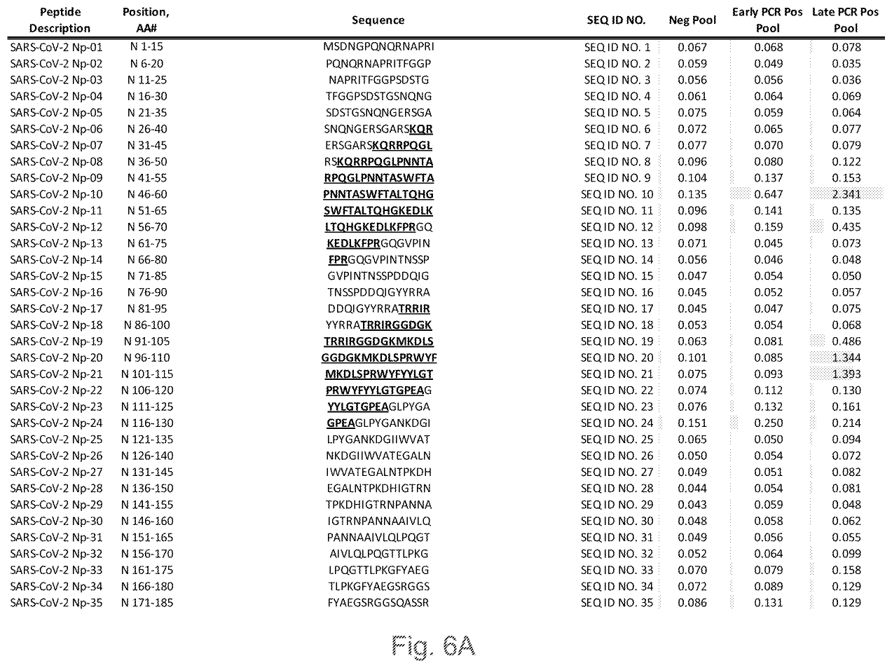

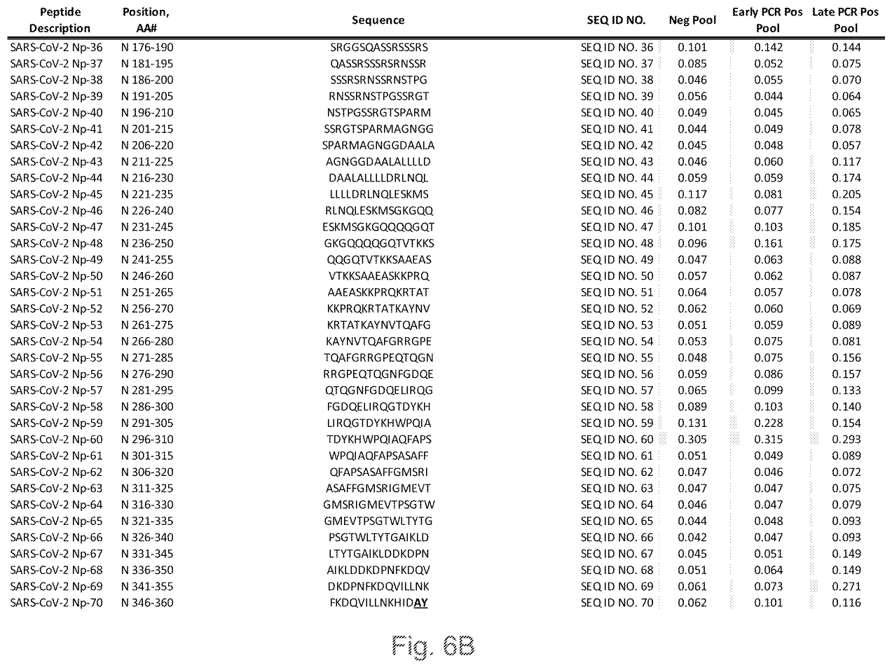

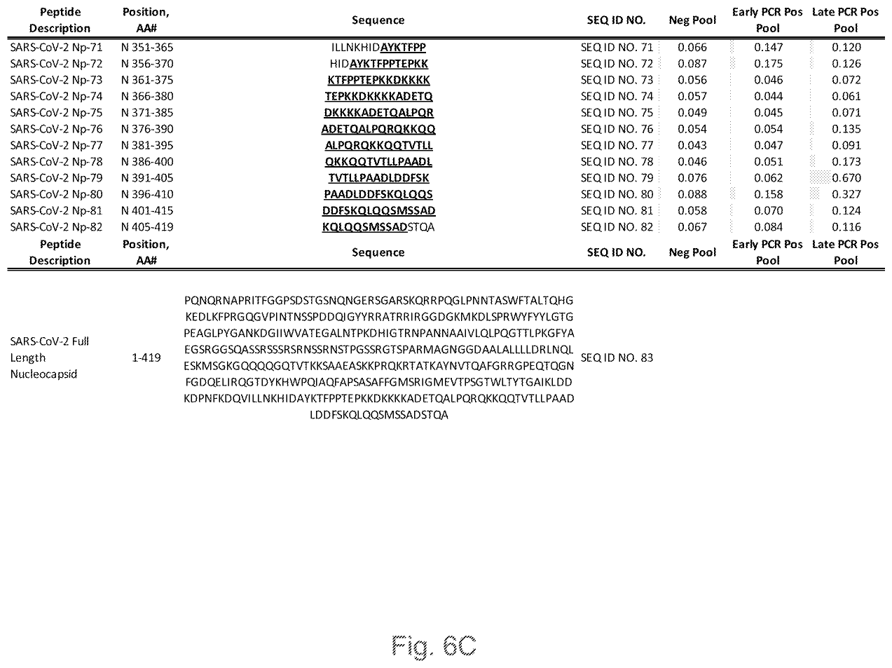

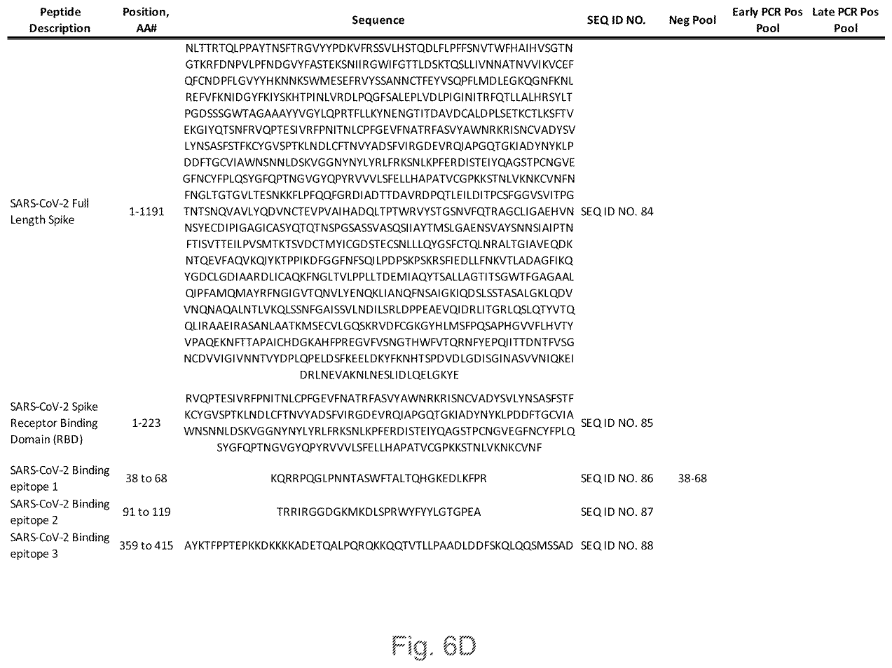

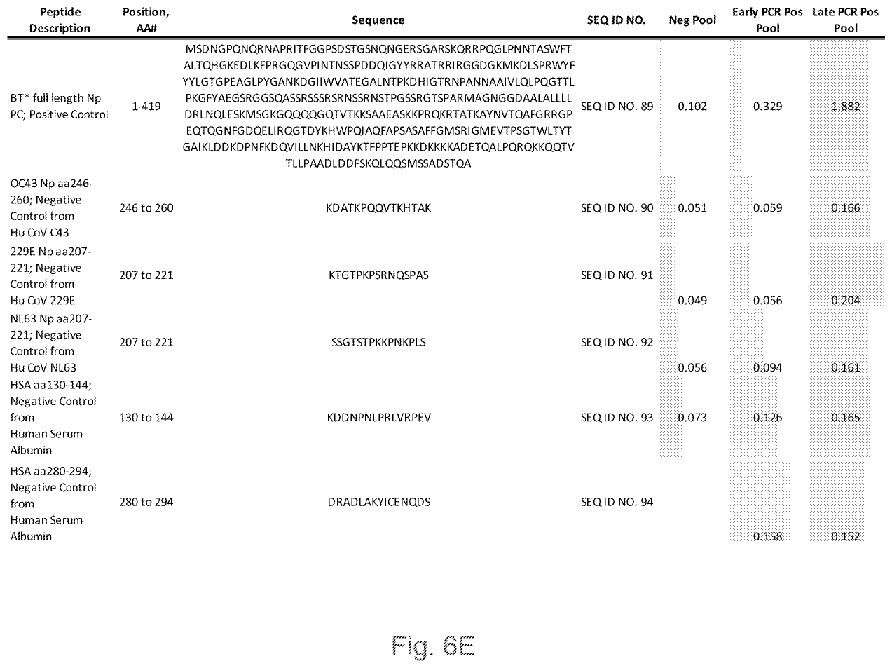

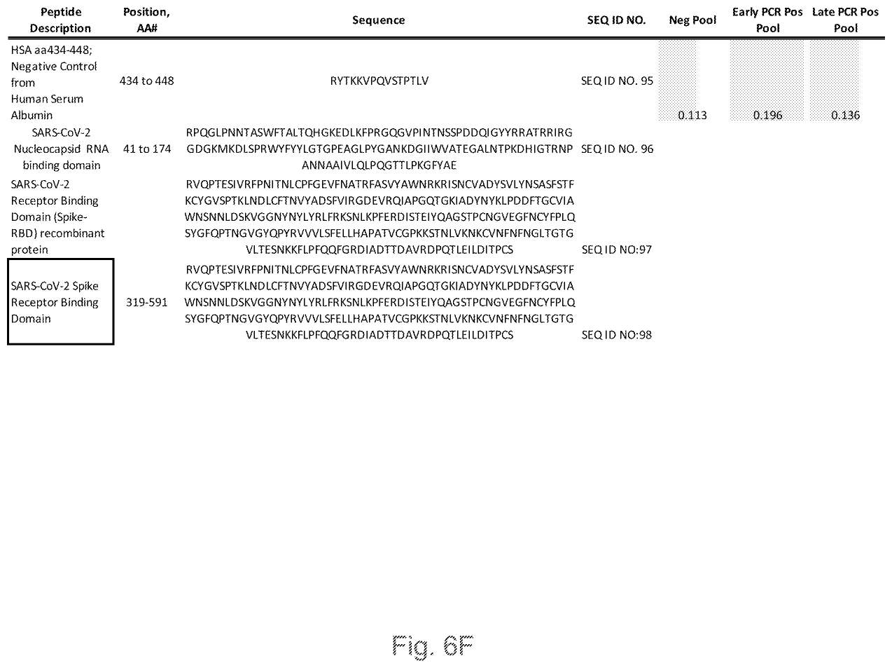

The results (FIG. 6) demonstrate the presence of multiple linear immunodominant epitopes within the SARS-CoV-2 Nucleocapsid protein sequence as indicated by the positive patient seroreactivity observed for multiple peptides including but not limited to: Np-10 (aa 46-60); Np-20 & Np-21 (aa 96-115); and Np-79 (aa 391-405).

### Example 3

### Dual Antigen Assay

Serum samples from four known SARS-CoV2 antibody positive patients were used m a serial dilution series (neat (undiluted), 1;2, 1:4, 1:8, 1:16, 1:32, 1;64, 1;128, 1:256, 1:512, 1:1024, and 1:2048). Fetal bovine serum (FBS) was used as a negative control.

Plate coating. Streptavidin coating of microtiter plate wells. 100 μl of streptavidin solution (5 μg/ml in 0.05M borate buffer (pH 9.5)) was added to each well and incubated overnight at room temperature (RT). The next day plates were washed 2 times with 300 μl/well of wash block solution (50 mM phosphate buffered saline (PBS), 0.15% w/v Tyloxapol). 200 μl of overcoat solution (50 mM PBS, 0.15% w/v Tyloxapol, 2.5% w/v sucrose) was dispensed to each well and immediately aspirated. Plates were put in a vacuum plate dryer for 4 hours to dry. Biotinylated spike receptor binding domain (Spike-RBD) or the RNA binding domain of nucleocapsid protein (Np) were diluted in PBS at 3 μg/ml and 2 μg/ml, respectively. 100 μl of biotinylated antigen solution were added to streptavidin plates for 1 hour at RT. Plates were wash blocked with 200 μl/well sucrose overcoat (PBS-T (50 mM PBS, 0.1% v/v Tweeu-20) with 2.5% sucrose). Sucrose solution was aspirated and plates dried for 4 hours in a vacuum plate dryer.

Assay. ELISA detection reagents comprised either Spike-RBD, Np RNA binding domain, or both Spike-RBD and Np RNA binding domain antigens conjugated to HRP (0.25 μl/ml). The assay protocol included the following steps: (1) mixing sample with one (either Spike-RBD or Np RNA binding domain) or both conjugates (Spike-RBD and Np RNA binding domain) and incubating on coated plates for one hour to create a dual antigemantibody sandwich; (2) washing and aspirating plates to remove unbound reagents; (3) adding TMB substrate (1.2 mM 3,3′,5,5′-Tetramethylbenzidine, 3.0 mM hydrogen superoxide/peroxide; Seramun Diagnostica, GmbH) and incubate for 15 minutes; (4) adding maleic acid stop solution (40 g/L maleic acid, 0.25 ml/L Proclin 300) and reading the plate at 450 nm to measure color development in each well.

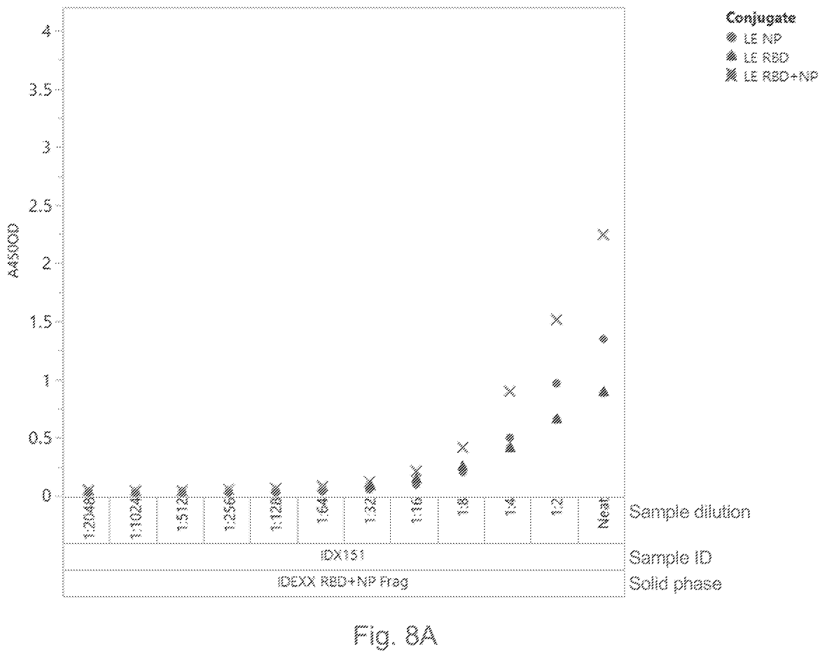

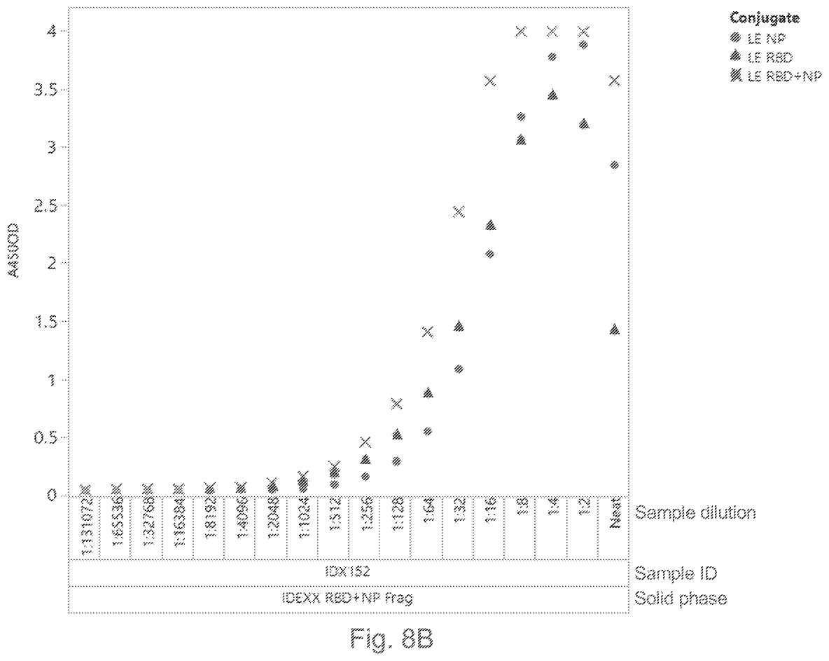

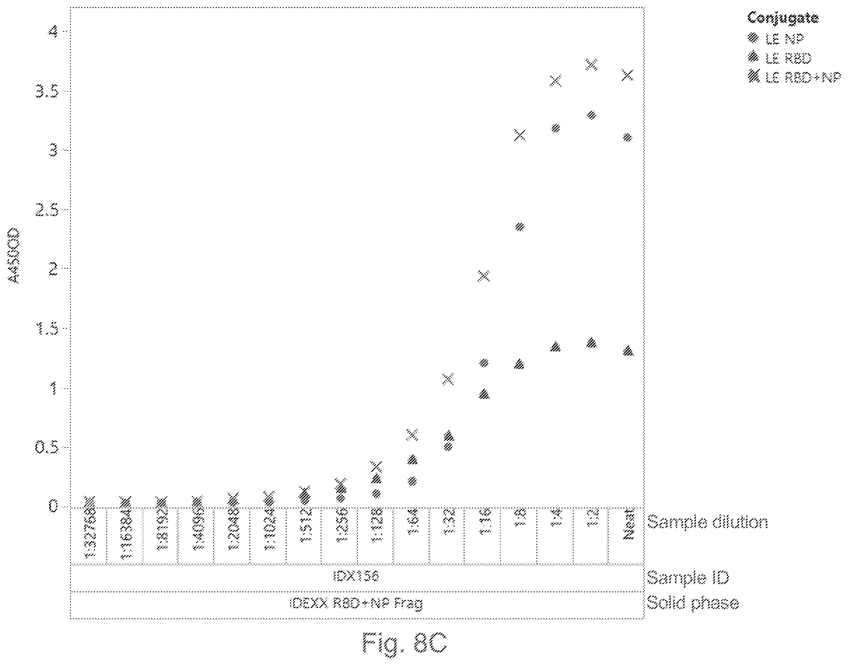

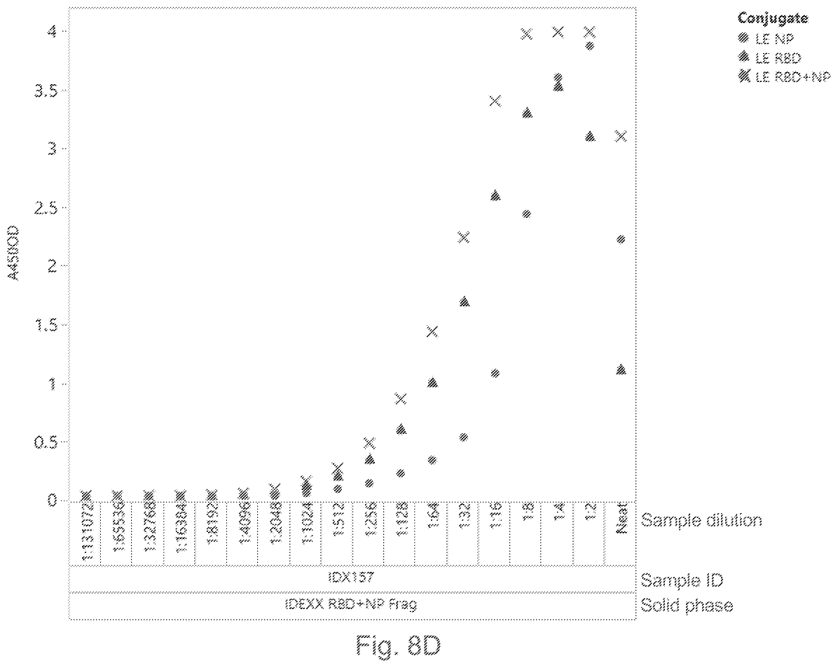

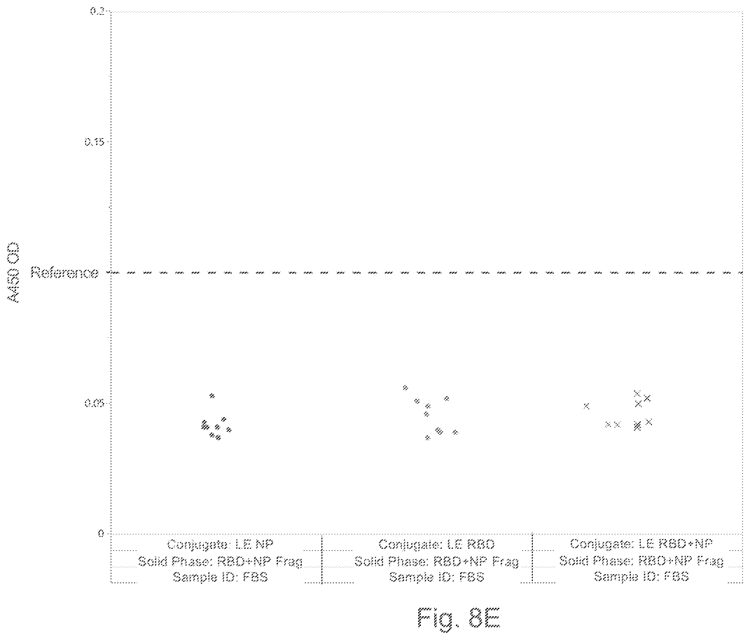

Results from the SARS-CoV2 antibody positive sera are shown in FIGS. 8A-D. The analytical sensitivity of the dual antigen assay (LE RBD+Np) was increased by approximately 1 dilution factor (i.e., 2-fold) from that of the single antigen (RBD assay). The dual antigen assay changed the kinetics of prozoning (i.e., the portion of die range of concentration of antibody-antigen mixtures in which one of them, although present in excess, does not produce its characteristic effect); for instance, sample IDX152 began to prozone on the RBD assay when going from a 1:4 to a 1:2 dilution, while the dual antigen assay showed no loss of signal between those two dilutions. Also, the loss of signal from prozoning was smaller in the dual antigen assay than in the single antigen assays, FIG. 8E shows protein levels for RDB, Np and RDB+Np were essentially equivalent.

### Example 4A

### Detection of SARS-CoV-2-Specific Total Antibodies to Spike-RBD by ASA

An ELISA assay for the detection of total antibodies (IgO, IgM, and IgA) against the Spike-RBD domain of SARS-CoV-2 was made as follows. SARS-CoV-2 Receptor Binding Domain (Spike-RBD) recombinant protein (SEQ ID NO:97) was coated onto microtiter plates. A horseradish peroxidase conjugate of the SARS-CoV-2 Receptor Binding Domain (Spike-RBD) protein (SEQ ID NO:98) was used as the assay detector. Serum or plasma samples were diluted 1:2 μL with the Spike-RBD-HRP conjugate by diluting 60 μL of sample with 60 μL of the conjugate. 100 μL of diluted sample was dispensed into each appropriate well of the microtiter plates containing the immobilized Spike-RBD and incubated for 60 minutes at 18-25° C. If present, SARS-CoV-2 antibody/Spike-RBD-HRP complexes bound to the immobilized Spike-RBD. The solution was removed, and each was washed with approximately 300 μL of wash solution (PBS; 0.0016 g/L (0.00016%) gentamicin; and 0.71 g/L (0.075%) of a zwitterionic detergent (e.g., N-tetradecyl-N,N-dimethyl-3-ammonio-1-propanesulfonate or 3-(N,N-Dimethyltetradecylammonio)propanesulfon)) 5 times. Each plate was tapped onto absorbent material after the final wash to remove any residual wash solution. TMB substrate was added and reacted with the HRP of any bound complexes to generate a blue color. The color reaction was then stopped with the addition of maleic acid stop solution, shifting color from blue to yellow. Optical densities (A450 nm) were, read and results were calculated by generating a sample to positive control ratio (S/P sample to positive ratio was calculated by using the absorbance obtained with the test sample and a positive control (A450 nm), corrected for the absorbance of the negative control. The positive control contains an anti-SARS-CoV-2 Receptor Binding Domain (Spike-RBD) antibody. Color development indicated the presence of anti-SARS-CoV-2 antibodies in the test sample.

SEQ ID NOS:97 and 98 were expressed, isolated and purified utilizine a C-terminal HIS-tag with a TEV protease cleavage site according to methods known in the art.

### Example 4B

### Assessment of Cross-Reactivity

The disease-state samples listed in Table 1 were tested on the ELISA assay described in Example 4A to assess cross-reactivity. One (1) sample out of 108 yielded a positive result. The results are summarized in Table 1.

TABLE 1

| Clinical Condition | Number tested | Number Positive |
|---|---|---|
| Human Coronavirus OC43 | 12 | 0 |
| Human Coronavirus 229E | 12 | 0 |
| Human Coronavirus HKU1 | 12 | 0 |
| Human Coronavirus NL63 | 12 | 0 |
| Haemophilus influenza | 7 | 0 |
| Mycoplasma pneumoniae | 4 | 0 |
| anti-Influenza A IgG | 5 | 1 |
| anti-Influenza B IgG | 5 | 0 |
| anti-Respiratory Syncytial | 5 | 0 |
| Virus IgG | | |
| anti-Hepatitis A Virus | 5 | 0 |
| anti-Hepatitis B Virus | 5 | 0 |
| anti-Hepatitis C Virus | 5 | 0 |
| HIV Seropositive | 10 | 0 |
| RF | 5 | 0 |
| Antinuclear Antobodies | 4 | 0 |
| (ANA) | | |
| Total | 108 | 1 |

### Example 4C

### Impact of Potentially Interfering Substances

Antibody-negative and positive SQRS-CoV-2 samples spanning the test dynamic range were spiked with the following materials, at noted concentrations, and to on the ELISA assay described in Example 4A. As shown in Table 2, no false positives or false negatives were observed.

TABLE 2

| | | Positive | | Positive | | Positive | | Negative | | Negative | |
|---|---|---|---|---|---|---|---|---|---|---|---|
| Substance | Conc. | Sample | | Sample | | Sample | | Sample | | Sample | |
| Tested | (mg/mL) | S/P | Result | S/P | Result | S/P | Result | S/P | Result | S/P | Result |
| Control | NA | 1.66 | Pos. | 0.92 | Pos. | 0.58 | Pos. | 0.01 | Neg. | 0.01 | Neg. |
| Cholesterol | 30 | 1.66 | Pos. | 1.10 | Pos. | 0.62 | Pos. | 0.01 | Neg. | 0.00 | Neg. |
| Hemoglobin | 10 | 1.50 | Pos. | 0.96 | Pos. | 0.54 | Pos. | 0.01 | Neg. | 0.09 | Neg. |
| Bilirubin | 0.4 | 1.78 | Pos. | 0.90 | Pos. | 0.54 | Pos. | 0.01 | Neg. | 0.01 | Neg. |

### Example 4D

### Clinical Sensitivity/Positive Percent Agreement

The clinical sensitivity was determined by evaluating the ELISA assay described in Example 4A with samples collected from a total of 155 patients where the time between onset of symptoms and blood collection was noted and from 201 patients where time post PCR result was recorded.

The following Table 3 describes the clinical sensitivity by time of sampling post Onset of Symptoms:

TABLE 3

| Days from | Total PCR | | | | |
|---|---|---|---|---|---|
| Onset of | Positive | Number | Number Non- | | |
| Symptoms | Samples | Reactive | Reactive | PPA | 95% CI |
| </=7 | 0 | 0 | 0 | NA | NA |
| 8-14 | 1 | 0 | 1 | 0% | −2.9%; 82.9% |
| >/=15 | 154 | 148 | 6 | 96.1% | 91.5%; 98.4% |
| Total Samples | 155 | | | | |

The following Table 4 describes the clinical sensitivity by time of sampling post PCR positive result:

TABLE 4

| Days from | Total PCR | | | | |
|---|---|---|---|---|---|
| Onset of | Positive | Number | Number Non- | | |
| Symptoms | Samples | Reactive | Reactive | PPA | 95% CI |
| </=7 | 2 | 2 | 0 | 100% | 28.9%; 100% |
| 8-14 | 9 | 8 | 1 | 88.9% | 54%; 99.8% |
| >/=15 | 190 | 184 | 6 | 96.8% | 93.1%; 98.7% |
| Total Samples | 201 | | | | |

### Example 4E

### Clinical Specificity/Negative Percent Agreement

The clinical specificity of the ELISA assay described in Example 4A was determined with samples collected in 2019, prior to the appearance of SARS-CoV-2. The rest are shown in Table 5.

TABLE 5

| | | | | | Negative | |
|---|---|---|---|---|---|---|
| | | | | | Percent | |
| | | Total | Number | Number | Agreement | |
| | Matrix | Samples | Reactive | Non-Reactive | (NPA) | 95% CI |
| Healthy | | | | | | |
| Donors | | | | | | |
| UK 2019 | Serum | 98 | 1 | 97 | 99.0% | 93.8%; 100% |
| UK 2019 | Plasma | 99 | 3 | 96 | 97.0% | 91%; 99.3% |
| USA 2019 #1 | Serum | 50 | 2 | 48 | 96.0% | 85.6%; 99.6% |
| USA 2019 #2 | Plasma | 50 | 1 | 49 | 98.0% | 88.3%; 100% |
| | Healthy Total | 297 | 7 | 290 | 97.6% | 95.1%; 98.9% |
| Clinical | | | | | | |
| Condition | | | | | | |
| 2019 | Various | 108 | 1 | 107 | 99.1% | 94.3%; 100% |
| Collections | | | | | | |

| | | | | | |
|---|---|---|---|---|---|
| Grand Total 2019 | 405 | 8 | 397 | 98.0% | 96.1%; 99.1% |

### Example 5

### Detection of SARS-CoV-2-Specific Antibodies in Saliva

A test was conducted to determine if the ELISA assay described in Example 4 is suitable for the detection of SARS-CoV-2-specific antibodies in saliva.

Saliva from fully, partially or unvaccinated individuals as collected using the Oracol Saliva Collection Device (Malvern Medical Developments Ltd., Worcester, UK. Product Code S10). The collection was conducted for 45 seconds on the top teeth and 45 seconds on the bottom teeth. The device was placed back into the tube upside down and ceritrifuged at 3000 g for 5 minutes to extract the fluid from the sponge. The device was carefully removed from the tube and discarded. The saliva supernatant was pipetted into a fresh tube while avoiding any dislodging of the pellet). The saliva supernatant was stored at −80 C. Prior to testing, the saliva supernatant slowly thawed on ice.

Before running the ELISA assay, aliquots of the saliva supernatants were pre-incubated with either PBS or recombinant Spike-RBD protein for 20 minutes at room temperature. During pre-incubation, Spike-RBD will bind to any anti-Spike-RBD antibodies present in the saliva supernatants, making the antibodies unavailable to bind to the plate, and thus will reduce the signal, demonstrating specificity.

The ELISA assay was conducted as described in Example 4 except that the plate was pre-blocked by adding 100 μl of conjugate solution to each well and incubating at room temperature for 10 minutes, followed by aspiration of the conjugate solution from the plate.

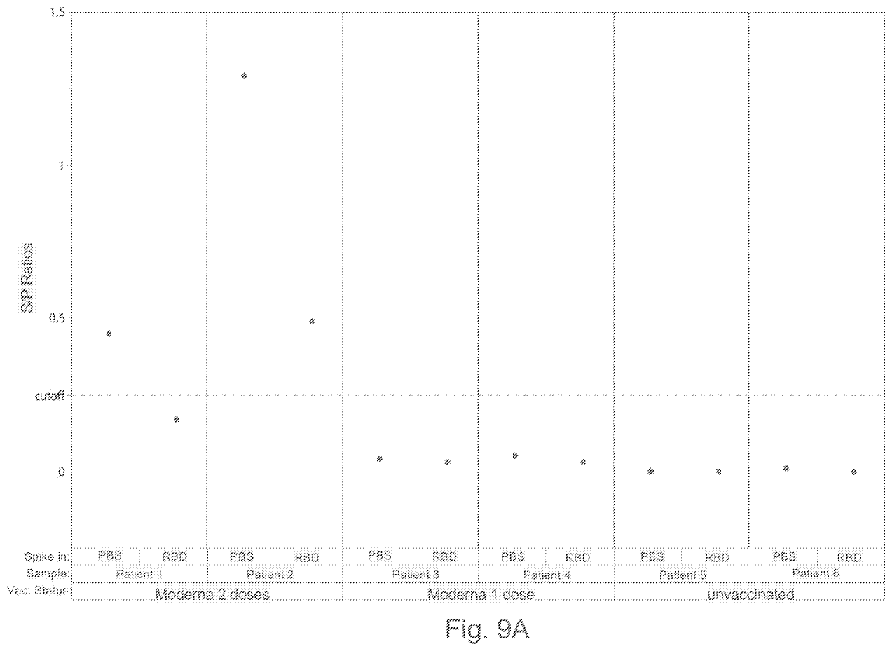

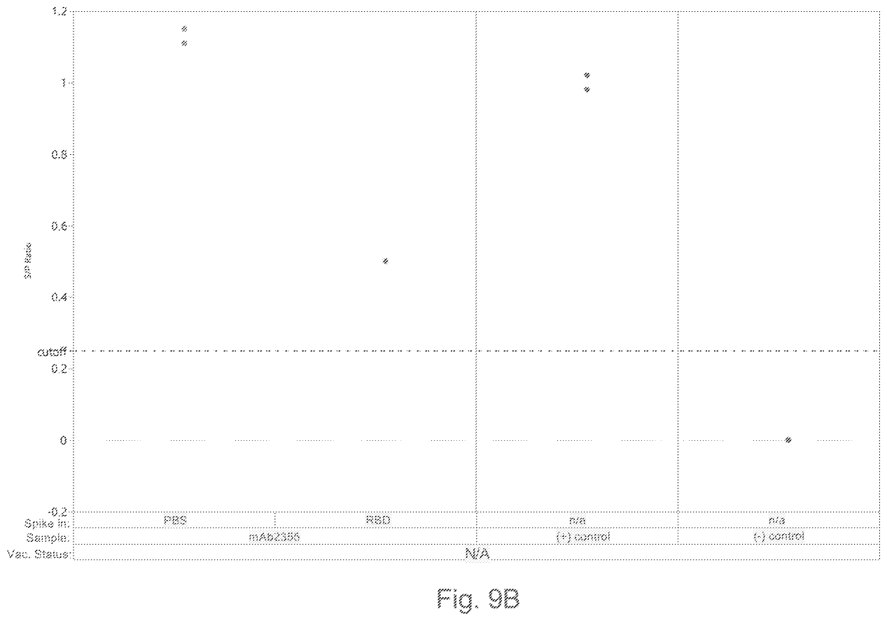

The results are shown in FIGS. 9A-B. Saliva from both fully vaccinated patients (Patient 1 and Patient 2) resulted in a positive S/P ratio. The signal was greatly diminished after pre-incubation with Spike-RBD protein, Saliva from both partially-vaccinated patients (Patient 3 and Patient 4 and both unvaccinated patients (Patient 5 and Patient 6) resulted in a S/P ratios below the detection threshold. PC and NC are positive mid negative controls. The sample “mAB-2355” is a specificity control. in which negative saliva was spiked with anti-RBD human antibodies and then pre-incubated with either PBS or recombinant Spike-RBD protein. Reduction of signal after pre-incubation with Spike-RBD protein demonstrates that anti-Spike-RBD antibodies are specifically removed by pre-incubation with Spike-RBD. This data demonstrates the ability, of the assay to specifically detect anti-Spike-RBD antibodies in human saliva.

### Example 6

### Detection of SARS-CoV-2-Specific Total Antibodies to Spike-RBD in Samples from Vaccinated Patients

Thirty (30) matched patient samples (Access Biologicals) were tested with the Diasorin Liaison test, which was used as the standard to determine the presence or absence of antibody titers to SARS-CoV-2. Samples were run on the assay described in Example 4.

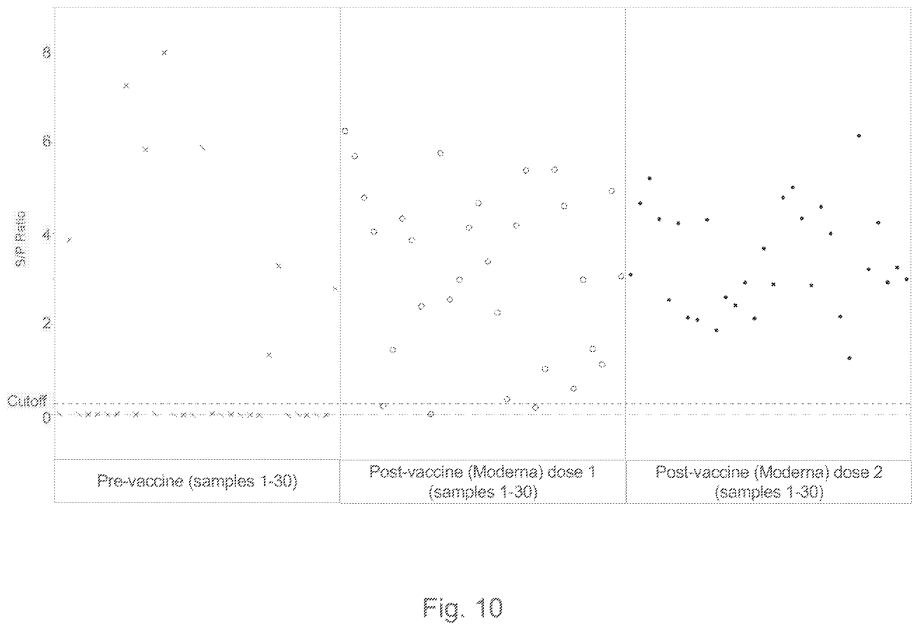

As shown in FIG. 10, the samples from all 30 vaccinated individuals were positive in the present test after dose 2 (detection rate=100%). The samples from 27 vaccinated individuals were positive in the present test after dose 1 (detection rate 90% (27/30)). Samples from six donors that showed a positive titer in their pre-vaccine sample, according to the Diasorin Liaison test, were all positive in the present test as well. A pre-vaccine sample from one donor had a negative titer according to the Diasorin Liaison test but was positive in the present test.

The examples given above are merely illustrative and are not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to the skilled artisan.

it is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention. It also is to be noted that, as used herein and in the appended claims, the singular forms “a,” “an,” and “the” include the plural reference unless the context clearly dictates otherwise. Thus, for example, a reference to “a linker” is a reference to one or more linkers and equivalents thereof known to those skilled in the art.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein.

Any numerical values recited herein include all values from the lower value to the upper value in increments of one unit provided that there is a separation of at least two units between any lower value and any higher value. As an example, if it is stated that the concentration of a component or value of a process variable such as, for example, size, angle size, pressure, time and the like, is, for example, from 1 to 90, specifically from 20 to 80, more specifically from 30 to 70, it is intended that values such as 15 to 85, 22 to 68, 43 to 51, 30 to 32, etc. are expressly enumerated in this specification. For values which are less than one, one unit is considered to be 0.0001, 0.001, 0,01 or 0.1 as appropriate. These are only examples of what is specifically intended and all possible combinations of numerical values between the lowest Value and the highest value enumerated are to be considered to be expressly stated in this application in a similar manner.

Particular methods, devices, and materials are described, although any methods and materials similar or equivalent to those described herein can be used in the practice or testing of the invention. The disclosures of all references and publications cited above are expressly incorporated by reference in their entireties to the same extent as if each were incorporated by reference individually,

TABLE 6
Sequences

| Peptide | Position, | | SEQ ID |
|---|---|---|---|
| Description | AA# | Sequence | NO. |
| SARS-CoV-2 | N 1-15 | MSDNGPQNQRNAPRI | SEQ ID |
| Np-01 | | | NO: 1 |
| SARS-CoV-2 | N 6-20 | PQNQRNAPRITFGGP | SEQ ID |
| Np-02 | | | NO: 2 |
| SARS-CoV-2 | N 11-25 | NAPRITFGGPSDSTG | SEQ ID |
| Np-03 | | | NO: 3 |
| SARS-CoV-2 | N 16-30 | TFGGPSDSTGSNQNG | SEQ ID |
| Np-04 | | | NO: 4 |
| SARS-CoV-2 | N 21-35 | SDSTGSNQNGERSGA | SEQ ID |
| Np-05 | | | NO: 5 |
| SARS-CoV-2 | N 26-40 | SNQNGERSGARSKQR | SEQ ID |
| Np-06 | | | NO: 6 |
| SARS-CoV-2 | N 31-45 | ERSGARSKQRRPQGL | SEQ ID |
| Np-07 | | | NO: 7 |
| SARS-CoV-2 | N 36-50 | RSKQRRPQGLPNNTA | SEQ ID |
| Np-08 | | | NO: 8 |
| SARS-CoV-2 | N 41-55 | RPQGLPNNTASWFTA | SEQ ID |
| Np-09 | | | NO: 9 |
| SARS-CoV-2 | N 46-60 | PNNTASWFTALTQHG | SEQ ID |
| Np-10 | | | NO: 10 |
| SARS-CoV-2 | N 51-65 | SWFTALTQHGKEDLK | SEQ ID |
| Np-11 | | | NO: 11 |
| SARS-CoV-2 | N 56-70 | LTQHGKEDLKFPRGQ | SEQ ID |
| Np-12 | | | NO: 12 |
| SARS-CoV-2 | N 61-75 | KEDLKFPRGQGVPIN | SEQ ID |
| Np-13 | | | NO: 13 |
| SARS-CoV-2 | N 66-80 | FPRGQGVPINTNSSP | SEQ ID |
| Np-14 | | | NO: 14 |
| SARS-CoV-2 | N 71-85 | GVPINTNSSPDDQIG | SEQ ID |
| Np-15 | | | NO: 15 |
| SARS-CoV-2 | N 76-90 | TNSSPDDQIGYYRRA | SEQ ID |
| Np-16 | | | NO: 16 |
| SARS-CoV-2 | N 81-95 | DDQIGYYRRATRRIR | SEQ ID |
| Np-17 | | | NO: 17 |
| SARS-CoV-2 | N 86-100 | YYRRATRRIRGGDGK | SEQ ID |
| Np-18 | | | NO: 18 |
| SARS-CoV-2 | N 91-105 | TRRIRGGDGKMKDLS | SEQ ID |
| Np-19 | | | NO: 19 |
| SARS-CoV-2 | N 96-110 | GGDGKMKDLSPRWYF | SEQ ID |
| Np-20 | | | NO: 20 |
| SARS-CoV-2 | N 101-115 | MKDLSPRWYFYYLGT | SEQ ID |
| Np-21 | | | NO: 21 |
| SARS-CoV-2 | N 106-120 | PRWYFYYLGTGPEAG | SEQ ID |
| Np-22 | | | NO: 22 |
| SARS-CoV-2 | N 111-125 | YYLGTGPEAGLPYGA | SEQ ID |
| Np-23 | | | NO: 23 |
| SARS-CoV-2 | N 116-130 | GPEAGLPYGANKDGI | SEQ ID |
| Np-24 | | | NO: 24 |
| SARS-CoV-2 | N 121-135 | LPYGANKDGIIWVAT | SEQ ID |
| Np-25 | | | NO: 25 |
| SARS-CoV-2 | N 126-140 | NKDGIIWVATEGALN | SEQ ID |
| Np-26 | | | NO: 26 |
| SARS-CoV-2 | N 131-145 | IWVATEGALNTPKDH | SEQ ID |
| Np-27 | | | NO: 27 |
| SARS-CoV-2 | N 136-150 | EGALNTPKDHIGTRN | SEQ ID |
| Np-28 | | | NO: 28 |
| SARS-CoV-2 | N 141-155 | TPKDHIGTRNPANNA | SEQ ID |
| Np-29 | | | NO: 29 |
| SARS-CoV-2 | N 146-160 | IGTRNPANNAAIVLQ | SEQ ID |
| Np-30 | | | NO: 30 |
| SARS-CoV-2 | N 151-165 | PANNAAIVLQLPQGT | SEQ ID |
| Np-31 | | | NO: 31 |
| SARS-CoV-2 | N 156-170 | AIVLQLPQGTTLPKG | SEQ ID |
| Np-32 | | | NO: 32 |
| SARS-CoV-2 | N 161-175 | LPQGTTLPKGFYAEG | SEQ ID |
| Np-33 | | | NO: 33 |
| SARS-CoV-2 | N 166-180 | TLPKGFYAEGSRGGS | SEQ ID |
| Np-34 | | | NO: 34 |
| SARS-CoV-2 | N 171-185 | FYAEGSRGGSQASSR | SEQ ID |
| Np-35 | | | NO: 35 |
| SARS-CoV-2 | N 176-190 | SRGGSQASSRSSSRS | SEQ ID |
| Np-36 | | | NO: 36 |
| SARS-CoV-2 | N 181-195 | QASSRSSSRSRNSSR | SEQ ID |
| Np-37 | | | NO: 37 |
| SARS-CoV-2 | N 186-200 | SSSRSRNSSRNSTPG | SEQ ID |
| Np-38 | | | NO: 38 |
| SARS-CoV-2 | N 191-205 | RNSSRNSTPGSSRGT | SEQ ID |
| Np-39 | | | NO: 39 |
| SARS-CoV-2 | N 196-210 | NSTPGSSRGTSPARM | SEQ ID |
| Np-40 | | | NO: 40 |
| SARS-CoV-2 | N 201-215 | SSRGTSPARMAGNGG | SEQ ID |
| Np-41 | | | NO: 41 |
| SARS-CoV-2 | N 206-220 | SPARMAGNGGDAALA | SEQ ID |
| Np-42 | | | NO: 42 |
| SARS-CoV-2 | N 211-225 | AGNGGDAALALLLLD | SEQ ID |
| Np-43 | | | NO: 43 |
| SARS-CoV-2 | N 216-230 | DAALALLLLDRLNQL | SEQ ID |
| Np-44 | | | NO: 44 |
| SARS-CoV-2 | N 221-235 | LLLLDRLNQLESKMS | SEQ ID |
| Np-45 | | | NO: 45 |
| SARS-CoV-2 | N 226-240 | RLNQLESKMSGKGQQ | SEQ ID |
| Np-46 | | | NO: 46 |
| SARS-CoV-2 | N 231-245 | ESKMSGKGQQQQGQT | SEQ ID |
| Np-47 | | | NO: 47 |
| SARS-CoV-2 | N 236-250 | GKGQQQQGQTVTKKS | SEQ ID |
| Np-48 | | | NO: 48 |
| SARS-CoV-2 | N 241-255 | QQGQTVTKKSAAEAS | SEQ ID |
| Np-49 | | | NO: 49 |
| SARS-CoV-2 | N 246-260 | VTKKSAAEASKKPRQ | SEQ ID |
| Np-50 | | | NO: 50 |
| SARS-CoV-2 | N 251-265 | AAEASKKPRQKRTAT | SEQ ID |
| Np-51 | | | NO: 51 |
| SARS-CoV-2 | N 256-270 | KKPRQKRTATKAYNV | SEQ ID |
| Np-52 | | | NO: 52 |
| SARS-CoV-2 | N 261-275 | KRTATKAYNVTQAFG | SEQ ID |
| Np-53 | | | NO: 53 |
| SARS-CoV-2 | N 266-280 | KAYNVTQAFGRRGPE | SEQ ID |
| Np-54 | | | NO: 54 |
| SARS-CoV-2 | N 271-285 | TQAFGRRGPEQTQGN | SEQ ID |
| Np-55 | | | NO: 55 |
| SARS-CoV-2 | N 276-290 | RRGPEQTQGNFGDQE | SEQ ID |
| Np-56 | | | NO: 56 |
| SARS-CoV-2 | N 281-295 | QTQGNFGDQELIRQG | SEQ ID |
| Np-57 | | | NO: 57 |
| SARS-CoV-2 | N 286-300 | FGDQELIRQGTDYKH | SEQ ID |
| Np-58 | | | NO: 58 |
| SARS-CoV-2 | N 291-305 | LIRQGTDYKHWPQIA | SEQ ID |
| Np-59 | | | NO: 59 |
| SARS-CoV-2 | N 296-310 | TDYKHWPQIAQFAPS | SEQ ID |
| Np-60 | | | NO: 60 |
| SARS-CoV-2 | N 301-315 | WPQIAQFAPSASAFF | SEQ ID |
| Np-61 | | | NO: 61 |
| SARS-CoV-2 | N 306-320 | QFAPSASAFFGMSRI | SEQ ID |
| Np-62 | | | NO: 62 |
| SARS-CoV-2 | N 311-325 | ASAFFGMSRIGMEVT | SEQ ID |
| Np-63 | | | NO: 63 |
| SARS-CoV-2 | N 316-330 | GMSRIGMEVTPSGTW | SEQ ID |
| Np-64 | | | NO: 64 |
| SARS-CoV-2 | N 321-335 | GMEVTPSGTWLTYTG | SEQ ID |
| Np-65 | | | NO: 65 |
| SARS-CoV-2 | N 326-340 | PSGTWLTYTGAIKLD | SEQ ID |
| Np-66 | | | NO: 66 |
| SARS-CoV-2 | N 331-345 | LTYTGAIKLDDKDPN | SEQ ID |
| Np-67 | | | NO: 67 |
| SARS-CoV-2 | N 336-350 | AIKLDDKDPNFKDQV | SEQ ID |
| Np-68 | | | NO: 68 |
| SARS-CoV-2 | N 341-355 | DKDPNFKDQVILLNK | SEQ ID |
| Np-69 | | | NO: 69 |
| SARS-CoV-2 | N 346-360 | FKDQVILLNKHIDAY | SEQ ID |
| Np-70 | | | NO: 70 |
| SARS-CoV-2 | N 351-365 | ILLNKHIDAYKTFPP | SEQ ID |
| Np-71 | | | NO: 71 |
| SARS-CoV-2 | N 356-370 | HIDAYKTFPPTEPKK | SEQ ID |
| Np-72 | | | NO: 72 |
| SARS-CoV-2 | N 361-375 | KTFPPTEPKKDKKKK | SEQ ID |
| Np-73 | | | NO: 73 |
| SARS-CoV-2 | N 366-380 | TEPKKDKKKKADETQ | SEQ ID |
| Np-74 | | | NO: 74 |
| SARS-CoV-2 | N 371-385 | DKKKKADETQALPQR | SEQ ID |
| Np-75 | | | NO: 75 |
| SARS-CoV-2 | N 376-390 | ADETQALPQRQKKQQ | SEQ ID |
| Np-76 | | | NO: 76 |
| SARS-CoV-2 | N 381-395 | ALPQRQKKQQTVTLL | SEQ ID |
| Np-77 | | | NO: 77 |
| SARS-CoV-2 | N 386-400 | QKKQQTVTLLPAADL | SEQ ID |
| Np-78 | | | NO: 78 |
| SARS-CoV-2 | N 391-405 | TVTLLPAADLDDFSK | SEQ ID |
| Np-79 | | | NO: 79 |
| SARS-CoV-2 | N 396-410 | PAADLDDFSKQLQQS | SEQ ID |
| Np-80 | | | NO: 80 |
| SARS-CoV-2 | N 401-415 | DDFSKQLQQSMSSAD | SEQ ID |
| Np-81 | | | NO: 81 |
| SARS-CoV-2 | N 405-419 | KQLQQSMSSADSTQA | SEQ ID |
| Np-82 | | | NO: 82 |
| SARS-CoV-2 | 1-419 | MSDNGPQNQRNAPRITFGGPSDSTGSNQNGERSG | SEQ ID |
| Full Length | | ARSKQRRPQGLPNNTASWFTALTQHGKEDLKFPR | NO: 83 |
| Nucleocapsid | | GQGVPINTNSSPDDQIGYYRRATRRIRGGDGKMK | |
| | | DLSPRWYFYYLGTGPEAGLPYGANKDGIIWVATE | |
| | | GALNTPKDHIGTRNPANNAAIVLQLPQGTTLPKGF | |
| | | YAEGSRGGSQASSRSSSRSRNSSRNSTPGSSRGTSP | |
| | | ARMAGNGGDAALALLLLDRLNQLESKMSGKGQ | |
| | | QQQGQTVTKKSAAEASKKPRQKRTATKAYNVTQ | |
| | | AFGRRGPEQTQGNFGDQELIRQGTDYKHWPQIAQ | |
| | | FAPSASAFFGMSRIGMEVTPSGTWLTYTGAIKLDD | |
| | | KDPNFKDQVILLNKHIDAYKTFPPTEPKKDKKKK | |
| | | ADETQALPQRQKKQQTVTLLPAADLDDFSKQLQ | |
| | | QSMSSADSTQA | |
| SARS-CoV-2 | 1-1191 | NLTTRTQLPPAYTNSFTRGVYYPDKVFRSSVLHST | SEQ ID |
| Full Length | | QDLFLPFFSNVTWFHAIHVSGTNGTKRFDNPVLPF | NO: 84 |
| Spike | | NDGVYFASTEKSNIIRGWIFGTTLDSKTQSLLIVNN | |
| | | ATNVVIKVCEFQFCNDPFLGVYYHKNNKSWMES | |
| | | EFRVYSSANNCTFEYVSQPFLMDLEGKQGNFKNL | |
| | | REFVFKNIDGYFKIYSKHTPINLVRDLPQGFSALEP | |
| | | LVDLPIGINITRFQTLLALHRSYLTPGDSSSGWTAG | |
| | | AAAYYVGYLQPRTFLLKYNENGTITDAVDCALDP | |
| | | LSETKCTLKSFTVEKGIYQTSNFRVQPTESIVRFPN | |
| | | ITNLCPFGEVFNATRFASVYAWNRKRISNCVADY | |
| | | SVLYNSASFSTFKCYGVSPTKLNDLCFTNVYADSF | |
| | | VIRGDEVRQIAPGQTGKIADYNYKLPDDFTGCVIA | |
| | | WNSNNLDSKYGGNYNYLYRLFRKSNLKPFERDIS | |
| | | TEIYQAGSTPCNGVEGFNCYFPLQSYGFQPTNGV | |
| | | GYQPYRVVVLSFELLHAPATVCGPKKSTNLVKNK | |
| | | CVNFNFNGLTGTGVLTESNKKFLPFQQFGRDIADT | |
| | | TDAVRDPQTLEILDITPCSFGGVSVITPGTNTSNQV | |
| | | AVLYQDVNCTEVPVAIHADQLTPTWRVYSTGSN | |
| | | VFQTRAGCLIGAEHVNNSYECDIPIGAGICASYQT | |
| | | QTNSPGSASSVASQSIIAYTMSLGAENSVAYSNNSI | |
| | | AIPTNFTISVTTEILPVSMTKTSVDCTMYICGDSTE | |
| | | CSNLLLQYGSFCTQLNRALTGIAVEQDKNTQEVF | |
| | | AQVKQIYKTPPIKDFGGFNFSQILPDPSKPSKRSFIE | |
| | | DLLFNKVTLADAGFIKQYGDCLGDIAARDLICAQ | |
| | | KFNGLTVLPPLLTDEMIAQYTSALLAGTITSGWTF | |
| | | GAGAALQIPFAMQMAYRFNGIGVTQNVLYENQK | |
| | | LIANQFNSAIGKIQDSLSSTASALGKLQDVVNQNA | |
| | | QALNTLVKQLSSNFGAISSVLNDILSRLDPPEAEV | |
| | | QIDRLITGRLQSLQTYVTQQLIRAAEIRASANLAA | |
| | | TKMSECVLGQSKRVDFCGKGYHLMSFPQSAPHG | |
| | | VVFLHVTYVPAQEKNFTTAPAICHDGKAHFPREG | |
| | | VFVSNGTHWFVTQRNFYEPQIITTDNTFVSGNCD | |
| | | VVIGIVNNTVYDPLQPELDSFKEELDKYFKNHTSP | |
| | | DVDLGDISGINASVVNIQKEIDRLNEVAKNLNESLI | |
| | | DLQELGKYE | |
| SARS-CoV-2 | 1-223 | RVQPTESIVRFPNITNLCPFGEVFNATRFASVYAW | SEQ ID |
| Spike Receptor | | NRKRISNCVADYSVLYNSASFSTFKCYGVSPTKLN | NO: 85 |
| Binding | | DLCFTNVYADSFVIRGDEVRQIAPGQTGKIADYN | |
| Domain (RBD) | | YKLPDDFTGCVIAWNSNNLDSKVGGNYNYLYRL | |
| | | FRKSNLKPFERDISTEIYQAGSTPCNGVEGFNCYFP | |
| | | LQSYGFQPTNGVGYQPYRVVVLSFELLHAPATVC | |
| | | GPKKSTNLVKNKCVNF | |
| SARS-CoV-2 | 38 to 68 | KQRRPQGLPNNTASWFTALTQHGKEDLKFPR | SEQ ID |
| Binding | | | NO: 86 |
| epitope 1 | | | |
| SARS-CoV-2 | 91 to 119 | TRRIRGGDGKMKDLSPRWYFYYLGTGPEA | SEQ ID |
| Binding | | | NO: 87 |
| epitope 2 | | | |
| SARS-Cov-2 | 359 to 415 | AYKTFPPTEPKKDKKKKADETQALPQRQKKQQT | SEQ ID |
| Binding | | VTLLPAADLDDFSKQLQQSMSSAD | NO: 88 |
| epitope 3 | | | |
| BT* | 1-419 | MSDNGPQNQRNAPRITFGGPSDSTGSNQNGERSG | SEQ ID |
| (biotinylated) | | ARSKQRRPQGLPNNTASWFTALTQHGKEDLKFPR | NO: 89 |
| full length Np | | GQGVPINTNSSPDDQIGYYRRATRRIRGGDGKMK | |
| PC; Positive | | DLSPRWYFYYLGTGPEAGLPYGANKDGIIWVATE | |
| Control | | GALNTPKDHIGTRNPANNAAIVLQLPQGTTLPKGF | |
| | | YAEGSRGGSQASSRSSSRSRNSSRNSTPGSSRGTSP | |
| | | ARMAGNGGDAALALLLLDRLNQLESKMSGKGQ | |
| | | QQQGQTVTKKSAAEASKKPRQKRTATKAYNVTQ | |
| | | AFGRRGPEQTQGNFGDQELIRQGTDYKHWPQIAQ | |
| | | FAPSASAFFGMSRIGMEVTPSGTWLTYTGAIKLDD | |
| | | KDPNFKDQVILLNKHIDAYKTFPPTEPKKDKKKK | |
| | | ADETQALPQRQKKQQTVTLLPAADLDDFSKQLQ | |
| | | QSMSSADSTQA | |
| OC43 Np | 246 to 260 | KDATKPQQVTKHTAK | SEQ ID |
| aa246-260; | | | NO: 90 |
| Negative | | | |
| Control from | | | |
| Hu CoV C43 | | | |
| 229E Np | 207 to 221 | KTGTPKPSRNQSPAS | SEQ ID |
| aa207-221; | | | NO: 91 |
| Negative | | | |
| Control from | | | |
| Hu CoV 229E | | | |
| NL63 Np | 207 to 221 | SSGTSTPKKPNKPLS | SEQ ID |
| aa207-221; | | | NO: 92 |
| Negative | | | |
| Control from | | | |
| Hu CoV NL63 | | | |
| HSA aal30- | 130 to 144 | KDDNPNLPRLVRPEV | SEQ ID |
| 144; Negative | | | NO: 93 |
| Control from | | | |
| Human Serum | | | |
| Albumin | | | |
| HSA aa280- | 280 to 294 | DRADLAKYICENQDS | SEQ ID |
| 294; Negative | | | NO: 94 |
| Control from | | | |
| Human Serum | | | |
| Albumin | | | |
| HSA aa434- | 434 to 448 | RYTKKVPQVSTPTLV | SEQ ID |
| 448; Negative | | | NO: 95 |
| Control from | | | |
| Human Serum | | | |
| Albumin | | | |
| SARS-CoV-2 | 41 to 174 | RPQGLPNNTASWFTALTQHGKEDLKFPRGQGVPI | SEQ ID |
| Nucleocapsid | | NTNSSPDDQIGYYRRATRRIRGGDGKMKDLSPRW | NO: 96 |
| RNA binding | | YFYYLGTGPEAGLPYGANKDGIIWVATEGALNTP | |
| domain | | KDHIGTRNPANNAAIVLQLPQGTTLPKGFYAE | |
| SARS-CoV-2 | | RVQPTESIVRFPNITNLCPFGEVFNATRFASVYAW | SEQ ID |
| Receptor | | NRKRISNCVADYSVLYNSASFSTFKCYGVSPTKLN | NO: 97 |
| Binding | | DLCFTNVYADSFVIRGDEVRQIAPGQTGKIADYN | |
| Domain | | YKLPDDFTGCVIAWNSNNLDSKVGGNYNYLYRL | |
| (Spike-RBD) | | FRKSNLKPFERDISTEIYQAGSTPCNGVEGFNCYFP | |
| recombinant | | LQSYGFQPINGVGYQPYRVVVLSFELLHAPATVC | |
| protein | | GPKKSTNLVKNKCVNFNFNGLTGTGVLTESNKKF | |
| | | LPFQQFGRDIADTTDAVRDPQTLEILDITPCS | |
| SARS-CoV-2 | 319-591 | RVQPTESIVRFPNITNLCPFGEVFNATRFASVYAW | SEQ ID |
| Spike Receptor | | NRKRISNCVADYSVLYNSASFSTFKCYGVSPTKLN | NO: 98 |
| Binding | | DLCFTNVYADSFVIRGDEVRQIAPGQTGKIADYN | |
| Domain | | YKLPDDFTGCVIAWNSNNLDSKVGGNYNYLYRL | |
| | | FRKSNLKPFERDISTEIYQAGSTPCNGVEGFNCYFP | |
| | | LQSYGFQPTNGVGYQPYRVVVLSFELLHAPATVC | |
| | | GPKKSTNLVKNKCVNFNFNGLTGTGVLTESNKKF | |
| | | LPFQQFGRDIADTTDAVRDPQTLEILDITPCS | |
| N38-Cap | | KQRRPQGLPNNTASWFTALTQHGKEDLKFPR | SEQ ID |
| | | | NO: 99 |
| N91-Cap | | TRRIRGGDGKMKDLSPRWYFYYLGTGPEA | SEQ ID |
| | | | NO: 100 |
| N359-Cap | | AYKTFPPTEPKKDKKKKADETQALPQRQKKQQ | SEQ ID |
| | | | NO: 101 |
| N384-Cap | | QRQKKQQTVTLLPAADLDDFSKQLQQSMSSAD | SEQ ID |
| | | | NO: 102 |
| N38-Det | | CKQRRPQGLPNNTASWFTALTQHGKEDLKFPR | SEQ ID |
| | | | NO: 103 |
| N91-Det | | CTRRIRGGDGKMKDLSPRWYFYYLGTGPEA | SEQ ID |
| | | | NO: 104 |
| N359-Det | | CAYKTFPPTEPKKDKKKKADETQALPQRQKKQQ | SEQ ID |
| | | | NO: 105 |
| N384-Det | | CQRQKKQQTVTLLPAADLDDFSKQLQQSMSSAD | SEQ ID |
| | | | NO: 106 |

What is claimed is:

1. A method for detecting a current or former SARS-CoV-2 infection in an animal, the method comprising:
determining a presence or amount of an antibody that hinds to a portion of a nucleocapsid polypeptide of SARS-CoV-2 in a sample from the animal; or
determining a presence or amount of an antibody that binds to a portion of a spike polypeptide of SARS-CoV-2 in the sample;
determining that the animal has a current or has had a previous SARS-CoV-2 infection by determining in the sample the presence or amount of at least one of the antibody that binds to a portion of nucleocapsid polypeptide or at least one of the antibody that binds to the portion of the spike polypeptide.

2. A method for detecting a current or former SARS-CoV-2 infection in an animal, the method comprising:
determining a presence or amount of an antibody that binds to a portion of a nucleocapsid polypeptide of SARS-CoV-2 in a sample from the animal;
determining a presence or amount of an antibody that binds to a portion of a spike polypeptide of SARS-CoV-2 in the sample;
determining that the animal has a current or has had a previous SARS-CoV-2 infection by determining in the sample the presence or amount of at least one of the antibody that binds to a portion of nucleocapsid polypeptide and at least one of the antibody that binds to the portion of the spike polypeptide.

3. The method of either one of claim 1 or 2, wherein the nucleocapsid polypeptide comprises SEQ ID NO:83.

4. The method of claim 3, wherein the portion of the nucleocapsid polypeptide comprises at least three consecutive amino acids from the RNA binding domain of the nucleocapsid polypeptide (SRQ ID NO:96).

5. The method of claim 4, wherein the portion of the nucleocapsid polypeptide comprises at. least three consecutive amino acids from one of SEQ ID NOS: 6-14, 17-24, 70-82, 86-88 and 99-106.

6. The method of either of claims 1 or 2, wherein the spike polypeptide comprises SEQ ID NO:84.

7. The method of claim 6, wherein the portion of the spike polypeptide comprises at least three amino acids from a receptor binding domain of the spike polypeptide.

8. The method of claim 7, wherein the receptor binding domain comprises SEQ NO:85.

9. A device for determining a current or former SARS-CoV-2 infection in an animal, the device comprising:
a solid phase having bound thereto a first polypeptide comprising a least a portion of a nucleocapsid polypeptide of SARS-CoV-2 or a second polypeptide comprising at least a portion of a spike poly-peptide of SARS-CoV-2.

10. A device for determining a current or former SARS-CoV-2 infection in an animal, the device comprising:
a solid phase having bound thereto a first polypeptide comprising a least a portion of a nucleocapsid polypeptide of SARS-CoV-2 and a second polypeptide comprising at least a portion of a spike polypeptide of SARS-CoV-2.

11. The device of either of claim 9 or 10, wherein the nucleocapsid polypeptide comprises SEQ ID NO:83.

12. The device of claim 11, wherein the portion of the nucleocapsid polypeptide comprises at least three consecutive amino acids from the RNA binding domain of the nucleocapsid polypeptide (SEQ ID NO:96).

13. The device of claim 12, wherein the portion of the nucleocapsid polypeptide comprises at least three consecutive amino acids from one of SEQ ID NOS: 6-14, 17-24, 70-82, 86-88 and 99-106.

14. The device of either one of claim 9 or 10, wherein the spike polypeptide comprises SEQ ID NO:84

15. The device of claim 14, wherein the portion of the spike polypeptide comprises at least three amino acids from a receptor binding domain of the spike polypeptide.

16. The device of claim 15, wherein the receptor binding domain comprises SEQ ID NO:85.

17. A kit for determining, a current or former SARS-CoV-2 infection in an animal, comprising the device of one of claims 9-16 and a conjugate comprising a labeled binding moiety that binds at least one a an antibody that binds to a nucleocapsid polypeptide of SARS-CoV-2 or at least one of an antibody that binds to a spike polypeptide of SARS-CoV-2.

18. A kit for determining a current or former SARS-CoV-2 infection in an animal, comprising the device of one of claims 9-16 and a conjugate comprising a labeled binding moiety that binds at least one of an antibody that binds to a nucleocapsid polypeptide of SARS-CoV-2 and at least one of an antibody, that binds to a spike polypeptide of SARS-CoV-2.

19. The kit of either one of claim 17 or 18, wherein the labeled binding moiety comprises an anti-species IgG antibody where the species is that of the animal.

20. A kit for determining a current or former SARS-CoV-2 infection in an animal, comprising
(a) the device of one of claims 9-16,
(b) first conjugate comprising a first labeled binding moiety that binds an antibody that binds to a nucleocapsid polypeptide of SARS-CoV-2, or
(c) a second conjugate comprising a labeled binding moiety that binds an antibody that binds to a spike polypeptide of SARS-CoV-2.

21. A kit for determining a current or former SARS-CoV-2 infection in an animal, comprising
(a) the device of one of claims 9-16,
(b) first conjugate comprising a first labeled binding moiety that binds at an antibody that binds to a nucleocapsid polypeptide of SARS-CoV-2, and
(c) a second conjugate comprising a labeled binding moiety that binds an antibody that binds to a spike polypeptide of SARS-CoV-2.

22. The kit of either one of claim 20 or 21, wherein the first conjugate comprises at least a portion of the nucleocapsid polypeptide.

23. The kit of claim 22, wherein the portion of the nucleocapsid polypeptide of the conjugate is the same as the portion of the nucleocapsid polypeptide bound to the solid phase.

24. The kit of either one of claim 20 or 21, wherein the second conjugate comprises at least a portion of the spike polypeptide.

25. The kit of claim 24, wherein the portion of the spike polypeptide of the conjugate is the same as the portion of the spike polypeptide bound to the solid phase.

26. A conjugate comprising at least consecutive three amino acids from a nucleocapsid polypeptide of SARS-CoV-2 and a detectable label.

27. The conjugate of claim 26, wherein the nucleocapsid polypeptide comprises SEQ ID NO:83.

28. The conjugate of claim 27, wherein the at least three amino acids are from the RNA binding domain of the nucleocapsid polypeptide (SEQ ID NO:96).

29. The conjugate of claim 26, wherein the at least three consecutive amino acids are from one of SEQ ID NOS: 6-14, 17-24, 70-82, 86-88 and 99-106.

30. A conjugate comprising at least three consecutive three amino acids from a spike polypeptide of SARS-CoV-2 and a detectable label.

31. The conjugate of claim 30 wherein the three consecutive amino acids are from the receptor binding domain of the spike polypeptide.

32. The conjugate of claim 30 wherein the spike polypeptide comprises SEQ. ID NO: 84

33. The conjugate of claim 31, wherein the receptor binding domain comprises SEQ ID NO:85.

34. A solid phase having bound thereto (a) a first immunological complex comprising an antibody that binds to a portion of a nucleocapsid polypeptide SARS-CoV-2 in a sample from the animal and the conjugate of one of claims 26-29, or (b) a second immunological complex comprising an antibody that binds to a portion of a spike polypeptide of SARS-CoV-2 in a sample from the animal and the conjugate of one of claims 30-33.

35. A solid phase having bound thereto (a) a first immunological complex comprising an antibody that binds to a portion of a nucleocapsid polypeptide of SARS-CoV-2 in a sample from the animal and the conjugate of one of claims 26-29, and (b) a second immunological complex comprising an antibody that binds to a portion of a spike polypeptide of SARS-CoV-2 in a sample from the animal and the conjugate of one of claims 30-33.

36. A polypeptide comprising at least three consecutive amino acids from one of SEQ ID NOS:6-14, 17-24, 70-82, 86-88 and 99-106. 37, A polypeptide comprising an amino acid sequence selected from the group consisting of any one of SEQ ID NOS 1-82, 86-88 and 99-106.

38. The polypeptide of claim 37 selected from the group consisting of SEQ ID NOS:6-14, 17-24, 70-82, 86-88, and 99-106.

39. The polypeptide of claim 38 selected from the group consisting of SEQ ID NOS:6-14, and 17-24.

40. The polypeptide of claim 38 selected from the group consisting of SEQ ID NOS: 70-82, and 86-88. 41, The polypeptide of claim 38 selected from the group consisting of SEQ ID NOS:99-106.

42. The polypeptide of any one of claims 36-41, wherein the polypeptide further comprises a detectable label.

43. The polypeptide of claim 42, wherein the label comprises one or more fluorescent label, isotopic label, biotin label, or enzyme conjugate label.

44. The polypeptide of any one of claims 36-41, wherein the polypeptide is reversibly or irreversibly bound to a solid support.

45. The polypeptide of any one of claims 36-41, wherein the polypeptide is attached at its N-terminus, its C-terminus or both termini to one or more other peptide sequences.

46. An immune complex comprising one or more polypeptides of any one of claims 36-45 and one or more antibodies that specifically hinds the one or more polypeptides, which one or more antibodies is from a sample from art animal suspected of having a SARS-CoV-2 infection.

47. A method of treating an animal infected with SARS-CoV-2, the method comprising:
determining a presence or amount of an antibody that binds to a portion of a nucleocapsid polypeptide of SARS-CoV-2 in a sample from the animal; or
determining a presence or amount of an antibody that binds to a portion of a spike polypeptide of BARS-CoV-2 in the sample:
determining that the animal has a SARS-CoV-2 infection by determining in the sample the presence or amount of at least one of the antibody that binds to a portion of nucleocapsid polypeptide or at least one the antibody that binds to the portion of the spike polypeptide;
administering an effective amount of a pharmaceutical composition to treat the SARS-CoV-2 infection, CoV-2 infection.

48. A method of treating an animal infected with SARS-CoV-2, the method comprising:
determining a presence or amount of an antibody that hinds to a portion of a nucleocapsid polypeptide of SARS-CoV-2 in a sample from the animal;
determining a presence or amount of an antibody that binds to a portion of a spike polypeptide of SARS-CoV-2 in the sample;
determining that the animal has a SARS-COV-2 infection by determining in the sample the presence or amount of at least one of the antibody that hinds to a portion of nucleocapsid polypeptide and at least one the antibody that binds to the portion of the spike polypeptide;
administering an effective amount of a pharmaceutical composition to treat the SARS-CoV-2 infection.

49. The method of either of claim 47 or 48, comprising determining in the sample the presence or amount of both the antibody that binds to a portion of nucleocapsid polypeptide and the antibody that, binds to the portion of the spike polypeptide.

50. The method of either of claim 47 or 48, wherein the animal has exhibited one or more symptoms of SARS-CoV-2 for no more than about 14, about 13 days, about 12 days, about 11 days, about 10 days; about 9 days, about 8 days, about 7 days, about 6 days, about 5 days, about 4 days, about 3 days, about 2 days, or about 1 day.

51. The method of claim 50, wherein the animal has exhibited one or more symptoms of SARS-CoV-2 for no more than about 10 days.

52. The method of claim either one of claim 47 or 48, wherein the pharmaceutical composition comprises one or more of antiviral drugs, corticosteroids, convalescent plasma, monoclonial antibodies, interleukin inhibitors, anti-parasitics, antibiotics, kinase inhibitors, interferons, and anti-inflammatories.

53. The method of claim 52, wherein antiviral drugs comprise one or more of remdesivir, lopinavir, ritonavir, darunavir, favipiravir, umifenovir, oseltamivir, disulfiram, danoprevir or nelfinavir, favipiravir, ribavirin, galidesivir, griffithsin, and nafamostat.

54. The method of claim 52, wherein anti-parasitics comprise one or more of hydroxychloroquine, chloroquine, and ivermectin.

55. The method of claims 52, wherein antibiotics comprise one or more of azithromycin, amoxicillin, clindamycin, cephalexin, ciprofloxacin, sulfamethoxazole/trimethoprim, metronidazole, levofloxacin, and doxycycline.

56. The method of claim 52, wherein monoclonal antibodies comprise one or more of bamlanivimab, etesevimab, casirivimab, imdevimab, S230.15, m396, S109.8 S227,14 S230.15, 80R scFv, CR3022 CR3014, 33G4 35B5, 30F9, 4D4, IF8, 5E9, B1 scFv, 47D11, HA001, B38, H-4, or CR3022.

57. The method of claim 52, wherein the interleukin inhibitors comprise one or more of tocilizumab sarilumab, anakinra, canakinumab, and rilonacept.

58. The method of claim 52, wherein the kinase inhibitors comprise one or more of acalabrutinib, baricitinib, ruxolitinib and tofacitinib.


SEQUENCE LISTING (106 sequences)

SEQ ID NO: 1 - protein, 15 aa, Artificial Sequence
  synthetic polypeptide
MSDNGPQNQRNAPRI

SEQ ID NO: 2 - protein, 15 aa, Artificial Sequence
  synthetic polypeptide
PQNQRNAPRITFGGP

SEQ ID NO: 3 - protein, 15 aa, Artificial Sequence
  synthetic polypeptide
NAPRITFGGPSDSTG

SEQ ID NO: 4 - protein, 15 aa, Artificial Sequence
  synthetic polypeptide
TFGGPSDSTGSNQNG

SEQ ID NO: 5 - protein, 15 aa, Artificial Sequence
  synthetic polypeptide
SDSTGSNQNGERSGA

SEQ ID NO: 6 - protein, 15 aa, Artificial Sequence
  synthetic polypeptide
SNQNGERSGARSKQR

SEQ ID NO: 7 - protein, 15 aa, Artificial Sequence
  synthetic polypeptide
ERSGARSKQRRPQGL

SEQ ID NO: 8 - protein, 15 aa, Artificial Sequence
  synthetic polypeptide
RSKQRRPQGLPNNTA

SEQ ID NO: 9 - protein, 15 aa, Artificial Sequence
  synthetic polypeptide
RPQGLPNNTASWFTA

SEQ ID NO: 10 - protein, 15 aa, Artificial Sequence
  synthetic polypeptide
PNNTASWFTALTQHG

SEQ ID NO: 11 - protein, 15 aa, Artificial Sequence
  synthetic polypeptide
SWFTALTQHGKEDLK

SEQ ID NO: 12 - protein, 15 aa, Artificial Sequence
  synthetic polypeptide
LTQHGKEDLKFPRGQ

SEQ ID NO: 13 - protein, 15 aa, Artificial Sequence
  synthetic polypeptide
KEDLKFPRGQGVPIN

SEQ ID NO: 14 - protein, 15 aa, Artificial Sequence
  synthetic polypeptide
FPRGQGVPINTNSSP

SEQ ID NO: 15 - protein, 15 aa, Artificial Sequence
  synthetic polypeptide
GVPINTNSSPDDQIG

SEQ ID NO: 16 - protein, 15 aa, Artificial Sequence
  synthetic polypeptide
TNSSPDDQIGYYRRA

SEQ ID NO: 17 - protein, 15 aa, Artificial Sequence
  synthetic polypeptide
DDQIGYYRRATRRIR

SEQ ID NO: 18 - protein, 15 aa, Artificial Sequence
  synthetic polypeptide
YYRRATRRIRGGDGK

SEQ ID NO: 19 - protein, 15 aa, Artificial Sequence
  synthetic polypeptide
TRRIRGGDGKMKDLS

SEQ ID NO: 20 - protein, 15 aa, Artificial Sequence
  synthetic polypeptide
GGDGKMKDLSPRWYF

SEQ ID NO: 21 - protein, 15 aa, Artificial Sequence
  synthetic polypeptide
MKDLSPRWYFYYLGT

SEQ ID NO: 22 - protein, 15 aa, Artificial Sequence
  synthetic polypeptide
PRWYFYYLGTGPEAG

SEQ ID NO: 23 - protein, 15 aa, Artificial Sequence
  synthetic polypeptide
YYLGTGPEAGLPYGA

SEQ ID NO: 24 - protein, 15 aa, Artificial Sequence
  synthetic polypeptide
GPEAGLPYGANKDGI

SEQ ID NO: 25 - protein, 15 aa, Artificial Sequence
  synthetic polypeptide
LPYGANKDGIIWVAT

SEQ ID NO: 26 - protein, 15 aa, Artificial Sequence
  synthetic polypeptide
NKDGIIWVATEGALN

SEQ ID NO: 27 - protein, 15 aa, Artificial Sequence
  synthetic polypeptide
IWVATEGALNTPKDH

SEQ ID NO: 28 - protein, 15 aa, Artificial Sequence
  synthetic polypeptide
EGALNTPKDHIGTRN

SEQ ID NO: 29 - protein, 15 aa, Artificial Sequence
  synthetic polypeptide
TPKDHIGTRNPANNA

SEQ ID NO: 30 - protein, 15 aa, Artificial Sequence
  synthetic polypeptide
IGTRNPANNAAIVLQ

SEQ ID NO: 31 - protein, 15 aa, Artificial Sequence
  synthetic polypeptide
PANNAAIVLQLPQGT

SEQ ID NO: 32 - protein, 15 aa, Artificial Sequence
  synthetic polypeptide
AIVLQLPQGTTLPKG

SEQ ID NO: 33 - protein, 15 aa, Artificial Sequence
  synthetic polypeptide
LPQGTTLPKGFYAEG

SEQ ID NO: 34 - protein, 15 aa, Artificial Sequence
  synthetic polypeptide
TLPKGFYAEGSRGGS

SEQ ID NO: 35 - protein, 15 aa, Artificial Sequence
  synthetic polypeptide
FYAEGSRGGSQASSR

SEQ ID NO: 36 - protein, 15 aa, Artificial Sequence
  synthetic polypeptide
SRGGSQASSRSSSRS

SEQ ID NO: 37 - protein, 15 aa, Artificial Sequence
  synthetic polypeptide
QASSRSSSRSRNSSR

SEQ ID NO: 38 - protein, 15 aa, Artificial Sequence
  synthetic polypeptide
SSSRSRNSSRNSTPG

SEQ ID NO: 39 - protein, 15 aa, Artificial Sequence
  synthetic polypeptide
RNSSRNSTPGSSRGT

SEQ ID NO: 40 - protein, 15 aa, Artificial Sequence
  synthetic polypeptide
NSTPGSSRGTSPARM

SEQ ID NO: 41 - protein, 15 aa, Artificial Sequence
  synthetic polypeptide
SSRGTSPARMAGNGG

SEQ ID NO: 42 - protein, 15 aa, Artificial Sequence
  synthetic polypeptide
SPARMAGNGGDAALA

SEQ ID NO: 43 - protein, 15 aa, Artificial Sequence
  synthetic polypeptide
AGNGGDAALALLLLD

SEQ ID NO: 44 - protein, 15 aa, Artificial Sequence
  synthetic polypeptide
DAALALLLLDRLNQL

SEQ ID NO: 45 - protein, 15 aa, Artificial Sequence
  synthetic polypeptide
LLLLDRLNQLESKMS

SEQ ID NO: 46 - protein, 15 aa, Artificial Sequence
  synthetic polypeptide
RLNQLESKMSGKGQQ

SEQ ID NO: 47 - protein, 15 aa, Artificial Sequence
  synthetic polypeptide
ESKMSGKGQQQQGQT

SEQ ID NO: 48 - protein, 15 aa, Artificial Sequence
  synthetic polypeptide
GKGQQQQGQTVTKKS

SEQ ID NO: 49 - protein, 15 aa, Artificial Sequence
  synthetic polypeptide
QQGQTVTKKSAAEAS

SEQ ID NO: 50 - protein, 15 aa, Artificial Sequence
  synthetic polypeptide
VTKKSAAEASKKPRQ

SEQ ID NO: 51 - protein, 15 aa, Artificial Sequence
  synthetic polypeptide
AAEASKKPRQKRTAT

SEQ ID NO: 52 - protein, 15 aa, Artificial Sequence
  synthetic polypeptide
KKPRQKRTATKAYNV

SEQ ID NO: 53 - protein, 15 aa, Artificial Sequence
  synthetic polypeptide
KRTATKAYNVTQAFG

SEQ ID NO: 54 - protein, 15 aa, Artificial Sequence
  synthetic polypeptide
KAYNVTQAFGRRGPE

SEQ ID NO: 55 - protein, 15 aa, Artificial Sequence
  synthetic polypeptide
TQAFGRRGPEQTQGN

SEQ ID NO: 56 - protein, 15 aa, Artificial Sequence
  synthetic polypeptide
RRGPEQTQGNFGDQE

SEQ ID NO: 57 - protein, 15 aa, Artificial Sequence
  synthetic polypeptide
QTQGNFGDQELIRQG

SEQ ID NO: 58 - protein, 15 aa, Artificial Sequence
  synthetic polypeptide
FGDQELIRQGTDYKH

SEQ ID NO: 59 - protein, 15 aa, Artificial Sequence
  synthetic polypeptide
LIRQGTDYKHWPQIA

SEQ ID NO: 60 - protein, 15 aa, Artificial Sequence
  synthetic polypeptide
TDYKHWPQIAQFAPS

SEQ ID NO: 61 - protein, 15 aa, Artificial Sequence
  synthetic polypeptide
WPQIAQFAPSASAFF

SEQ ID NO: 62 - protein, 15 aa, Artificial Sequence
  synthetic polypeptide
QFAPSASAFFGMSRI

SEQ ID NO: 63 - protein, 15 aa, Artificial Sequence
  synthetic polypeptide
ASAFFGMSRIGMEVT

SEQ ID NO: 64 - protein, 15 aa, Artificial Sequence
  synthetic polypeptide
GMSRIGMEVTPSGTW

SEQ ID NO: 65 - protein, 15 aa, Artificial Sequence
  synthetic polypeptide
GMEVTPSGTWLTYTG

SEQ ID NO: 66 - protein, 15 aa, Artificial Sequence
  synthetic polypeptide
PSGTWLTYTGAIKLD

SEQ ID NO: 67 - protein, 15 aa, Artificial Sequence
  synthetic polypeptide
LTYTGAIKLDDKDPN

SEQ ID NO: 68 - protein, 15 aa, Artificial Sequence
  synthetic polypeptide
AIKLDDKDPNFKDQV

SEQ ID NO: 69 - protein, 15 aa, Artificial Sequence
  synthetic polypeptide
DKDPNFKDQVILLNK

SEQ ID NO: 70 - protein, 15 aa, Artificial Sequence
  synthetic polypeptide
FKDQVILLNKHIDAY

SEQ ID NO: 71 - protein, 15 aa, Artificial Sequence
  synthetic polypeptide
ILLNKHIDAYKTFPP

SEQ ID NO: 72 - protein, 15 aa, Artificial Sequence
  synthetic polypeptide
HIDAYKTFPPTEPKK

SEQ ID NO: 73 - protein, 15 aa, Artificial Sequence
  synthetic polypeptide
KTFPPTEPKKDKKKK

SEQ ID NO: 74 - protein, 15 aa, Artificial Sequence
  synthetic polypeptide
TEPKKDKKKKADETQ

SEQ ID NO: 75 - protein, 15 aa, Artificial Sequence
  synthetic polypeptide
DKKKKADETQALPQR

SEQ ID NO: 76 - protein, 15 aa, Artificial Sequence
  synthetic polypeptide
ADETQALPQRQKKQQ

SEQ ID NO: 77 - protein, 15 aa, Artificial Sequence
  synthetic polypeptide
ALPQRQKKQQTVTLL

SEQ ID NO: 78 - protein, 15 aa, Artificial Sequence
  synthetic polypeptide
QKKQQTVTLLPAADL

SEQ ID NO: 79 - protein, 15 aa, Artificial Sequence
  synthetic polypeptide
TVTLLPAADLDDFSK

SEQ ID NO: 80 - protein, 15 aa, Artificial Sequence
  synthetic polypeptide
PAADLDDFSKQLQQS

SEQ ID NO: 81 - protein, 15 aa, Artificial Sequence
  synthetic polypeptide
DDFSKQLQQSMSSAD

SEQ ID NO: 82 - protein, 15 aa, Artificial Sequence
  synthetic polypeptide
KQLQQSMSSADSTQA

SEQ ID NO: 83 - protein, 419 aa, Artificial Sequence
  synthetic polypeptide
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 84 - protein, 1191 aa, Artificial Sequence
  synthetic polypeptide
NLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYE

SEQ ID NO: 85 - protein, 223 aa, Artificial Sequence
  synthetic polypeptide
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 86 - protein, 31 aa, Artificial Sequence
  synthetic polypeptide
KQRRPQGLPNNTASWFTALTQHGKEDLKFPR

SEQ ID NO: 87 - protein, 29 aa, Artificial Sequence
  synthetic polypeptide
TRRIRGGDGKMKDLSPRWYFYYLGTGPEA

SEQ ID NO: 88 - protein, 57 aa, Artificial Sequence
  synthetic polypeptide
AYKTFPPTEPKKDKKKKADETQALPQRQKKQQTVTLLPAADLDDFSKQLQQSMSSAD

SEQ ID NO: 89 - protein, 419 aa, Artificial Sequence; identical to SEQ ID NO: 83
  synthetic polypeptide
  MISC_FEATURE (38)..(38): X is native Lys (K) or biotinylated Lys (K-Biotin).
  MISC_FEATURE (61)..(61): X is native Lys (K) or biotinylated Lys (K-Biotin).
  MISC_FEATURE (65)..(65): X is native Lys (K) or biotinylated Lys (K-Biotin).
  MISC_FEATURE (100)..(100): X is native Lys (K) or biotinylated Lys (K-Biotin).
  MISC_FEATURE (102)..(102): X is native Lys (K) or biotinylated Lys (K-Biotin).
  MISC_FEATURE (127)..(127): X is native Lys (K) or biotinylated Lys (K-Biotin).
  MISC_FEATURE (143)..(143): X is native Lys (K) or biotinylated Lys (K-Biotin).
  MISC_FEATURE (169)..(169): X is native Lys (K) or biotinylated Lys (K-Biotin).
  MISC_FEATURE (233)..(233): X is native Lys (K) or biotinylated Lys (K-Biotin).
  MISC_FEATURE (237)..(237): X is native Lys (K) or biotinylated Lys (K-Biotin).
  MISC_FEATURE (248)..(248): X is native Lys (K) or biotinylated Lys (K-Biotin).
  MISC_FEATURE (249)..(249): X is native Lys (K) or biotinylated Lys (K-Biotin).
  MISC_FEATURE (256)..(256): X is native Lys (K) or biotinylated Lys (K-Biotin).
  MISC_FEATURE (257)..(257): X is native Lys (K) or biotinylated Lys (K-Biotin).
  MISC_FEATURE (261)..(261): X is native Lys (K) or biotinylated Lys (K-Biotin).
  MISC_FEATURE (266)..(266): X is native Lys (K) or biotinylated Lys (K-Biotin).
  MISC_FEATURE (299)..(299): X is native Lys (K) or biotinylated Lys (K-Biotin).
  MISC_FEATURE (338)..(338): X is native Lys (K) or biotinylated Lys (K-Biotin).
  MISC_FEATURE (342)..(342): X is native Lys (K) or biotinylated Lys (K-Biotin).
  MISC_FEATURE (347)..(347): X is native Lys (K) or biotinylated Lys (K-Biotin).
  MISC_FEATURE (355)..(355): X is native Lys (K) or biotinylated Lys (K-Biotin).
  MISC_FEATURE (361)..(361): X is native Lys (K) or biotinylated Lys (K-Biotin).
  MISC_FEATURE (369)..(369): X is native Lys (K) or biotinylated Lys (K-Biotin).
  MISC_FEATURE (370)..(370): X is native Lys (K) or biotinylated Lys (K-Biotin).
  MISC_FEATURE (372)..(375): X is native Lys (K) or biotinylated Lys (K-Biotin).
  MISC_FEATURE (387)..(387): X is native Lys (K) or biotinylated Lys (K-Biotin).
  MISC_FEATURE (388)..(388): X is native Lys (K) or biotinylated Lys (K-Biotin).
  MISC_FEATURE (405)..(405): X is native Lys (K) or biotinylated Lys (K-Biotin).

SEQ ID NO: 90 - protein, 15 aa, Artificial Sequence
  synthetic polypeptide
KDATKPQQVTKHTAK

SEQ ID NO: 91 - protein, 15 aa, Artificial Sequence
  synthetic polypeptide
KTGTPKPSRNQSPAS

SEQ ID NO: 92 - protein, 15 aa, Artificial Sequence
  synthetic polypeptide
SSGTSTPKKPNKPLS

SEQ ID NO: 93 - protein, 15 aa, Artificial Sequence
  synthetic polypeptide
KDDNPNLPRLVRPEV

SEQ ID NO: 94 - protein, 15 aa, Artificial Sequence
  synthetic polypeptide
DRADLAKYICENQDS

SEQ ID NO: 95 - protein, 15 aa, Artificial Sequence
  synthetic polypeptide
RYTKKVPQVSTPTLV

SEQ ID NO: 96 - protein, 134 aa, Artificial Sequence
  synthetic polypeptide
RPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAE

SEQ ID NO: 97 - protein, 273 aa, Artificial Sequence
  synthetic polypeptide
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCS

SEQ ID NO: 98 - protein, 273 aa, Artificial Sequence; identical to SEQ ID NO: 97
  synthetic polypeptide

SEQ ID NO: 99 - protein, 31 aa, Artificial Sequence; identical to SEQ ID NO: 86
  synthetic polypeptide

SEQ ID NO: 100 - protein, 29 aa, Artificial Sequence
  synthetic polypeptide
TRRIRGGDGKMKDLSPRWYFYYLGTGPEA

SEQ ID NO: 101 - protein, 32 aa, Artificial Sequence
  synthetic polypeptide
AYKTFPPTEPKKDKKKKADETQALPQRQKKQQ

SEQ ID NO: 102 - protein, 32 aa, Artificial Sequence
  Synthetic polypeptide
QRQKKQQTVTLLPAADLDDFSKQLQQSMSSAD

SEQ ID NO: 103 - protein, 32 aa, Artificial Sequence
  Synthetic polypeptide
CKQRRPQGLPNNTASWFTALTQHGKEDLKFPR

SEQ ID NO: 104 - protein, 30 aa, Artificial Sequence
  Synthetic polypeptide
CTRRIRGGDGKMKDLSPRWYFYYLGTGPEA

SEQ ID NO: 105 - protein, 33 aa, Artificial Sequence
  Synthetic polypeptide
CAYKTFPPTEPKKDKKKKADETQALPQRQKKQQ

SEQ ID NO: 106 - protein, 33 aa, Artificial Sequence
  Synthetic polypeptide
CQRQKKQQTVTLLPAADLDDFSKQLQQSMSSAD